(19) United States
(12) Patent Application Publication
(10) Pub. No.: US 2020/0306364 A1
(43) Pub. Date: Oct. 1, 2020

(54) MUCOADHESIVE NANOPARTICLE ENTRAPPED INFLUENZA VIRUS VACCINE DELIVERY SYSTEM

(71) Applicant: Ohio State Innovation Foundation, Columbus, OH (US)

(72) Inventors: Renukaradhya GOURAPURA, Wooster, OH (US); Santosh DHAKAL, Baltimore, MD (US); Sankar RENU, Wooster, OH (US)

(21) Appl. No.: 16/768,544

(22) PCT Filed: Nov. 30, 2018
(86) PCT No.: PCT/US2018/063299

§ 371 Date: May 29, 2020

Related U.S. Application Data
(60) Provisional application No. 62/592,869, filed on Nov. 30, 2017.

(51) Int. Cl. A61K 39/145

(52) U.S. Cl. CPC A61K 39/145; A61K 2039/5252

(57) ABSTRACT

Disclosed herein are nanoparticles comprising chitosan and an inactivated influenza A virus (IAV) antigen, wherein the chitosan encapsulates the inactivated IAV antigen. In some embodiments, the nanoparticle further comprises tripolyphosphate. In some embodiments, the nanoparticle reduces nasal shedding of an influenza A virus. In some embodiments, the nanoparticle elicits an increased amount of IgA antibody in a subject. Also disclosed are methods of reducing transmission of an influenza A virus, and methods of eliciting an immune response against an influenza A virus, in a subject compared to a control comprising administering to the subject a nanoparticle comprising chitosan and an inactivated influenza A virus (IAV) antigen, wherein the chitosan encapsulates the inactivated IAV antigen.

### CROSS REFERENCE TO RELATED APPLICATIONS

This application claims the benefit of U.S. Provisional Patent Application Ser. No. 62/592,869 filed Nov. 30, 2017, which is expressly incorporated herein by reference.

### STATEMENT REGARDING FEDERALLY SPONSORED RESEARCH

This invention was made with government support under Grant No. 2013-67015-20476 awarded by the United States Department of Agriculture National Institute of Food and Agriculture. The government has certain rights in the invention.

### FIELD

The disclosure generally relates to nanoparticles, particularly nanoparticles useable in a vaccine. The nanoparticles are capable of increasing immunity to various influenza A viruses. The disclosure further relates to methods to make and use nanoparticles and vaccines comprising nanoparticles.

### BACKGROUND

Swine influenza is an acute respiratory infection of pigs caused by influenza A virus (IAV) of the Orthomyxoviridae family, which possesses the negative sense segmented RNA genome. Virulent swine IAV (SwIAV) infection leads to acute febrile respiratory disease, which is often complicated with secondary bacterial infection. Acute clinical signs in influenza infected pigs include high fever, anorexia, respiratory distress, nasal discharge and coughing. Influenza causes significant economic loss in the pig industry through morbidity, loss of body weight gain, increased time to market, susceptibility to secondary bacterial and viral infections like mycoplasma and porcine reproductive and respiratory syndrome (PRRS), medication and veterinary expenses.

SwIAV increases its genetic diversity through frequent antigenic drift and antigenic shift. At present H1N1, H1N2 and H3N2 subtypes of IAV cause majority of infection in pigs. Owing to the presence of both avian (α2,3 Gal) and human (α2,6 Gal) IAV receptors, pigs can be infected with IAVs from different hosts and potentially act as mixing vessel for different IAVs. This favors genetic assortment and adaptation of novel influenza strain with zoonotic and even pandemic potential. Some SwIAV can be transmitted from pigs to humans creating public health risk. For example, the 2009 H1N1 swine influenza virus infected approximately 20% of the global population and caused around 200,000 deaths, in addition to the approximately 500,000 deaths due to seasonal annual influenza infection. The more recent H3N2 variant virus in the US is another example of swine origin IAVs in humans.

Controlling influenza in pigs through vaccination can serve the dual purpose of protecting economic loss in swine industry and preventing the possible public health risk. Swine influenza vaccines are commercially available and currently used in pigs. Predominantly, these are multivalent whole inactivated virus (WIV) vaccines given through the intramuscular route. Due to high mutation rates in circulating influenza viruses in animals, the efficacy of commercial vaccines in the field is usually poor. WIV vaccines provide protection against homologous viruses but fail to provide adequate heterologous immunity against IAVs constantly evolving through point mutations. The intramuscular-delivered WIV vaccines elicit a poor mucosal immune response, which is essential for providing cross-protective immunity against a multitude of variant IAVs. Thus, the persistent economic burden of swine influenza in the pig industry and its potential risk of zoonotic transmission to humans warrant the development of broadly cross-protective vaccine platforms.

Vaccination through the intranasal route can be a beneficial alternative to the current practice of intramuscular vaccination in pigs. Such mucosal vaccination not only induces better immune response at the local mucosal site, but also enhances immunity at distal mucosal sites and systemically.

Use of biodegradable and biocompatible polymer-based nanoparticle (NP) formulations can be an innovative strategy for vaccine antigen delivery to mucosal sites. Particulate vaccines facilitate antigen uptake by antigen presenting cells, maintain slow and sustained antigen release, prevent antigen from enzymatic degradation, and provide immunomodulatory properties. Inactivated/killed SwIAV antigen (KAg) encapsulated in poly(lactide co-glycolide) (PLGA) polymer based nanoparticles (NP) and delivered intranasal to pigs induced robust cross-reactive cell-mediated immune response associated with significant clearance of challenged heterologous virus from the lungs. Similarly, encapsulation of KAg in polyanhydride polymer-based NP also enhanced the cell-mediated response against SwIAV. However, both PLGA and polyanhydride polymer based intranasal vaccines failed to elicit mucosal IgA antibody response, probably due to their strongly biased ability to induce T helper 1 (Th1) response. As such, PLGA and polyanhydride polymer based intranasal vaccines failed to reduce nasal virus shedding in pigs. This is problematic because nasal virus shedding facilitates transmission between animals (e.g., pig to pig transmission, pig to human transmission).

The compositions and methods disclosed herein address these and other shortcomings in the art.

### SUMMARY

The disclosure herein addresses needs in the art by providing compositions and methods useful in the prevention and treatment of certain infectious agents. More specifically, the nanoparticles disclosed herein are capable of eliciting strong mucosal IgA responses and cellular immunity responses in the respiratory tract against influenza viruses. The nanoparticles provide robust immunity to influenza A viruses (IAV) and, importantly, reduce transmission of IAVs by reducing nasal viral shedding. The nanoparticles are particularly useful in vaccines administered to pigs to prevent or treat IAV infection.

In one aspect, disclosed herein is a nanoparticle comprising chitosan and an inactivated influenza A virus (IAV) antigen, wherein the chitosan encapsulates the inactivated IAV antigen. In some embodiments, the nanoparticle further comprises tripolyphosphate. In some embodiments, the nanoparticle reduces nasal shedding of an influenza A virus (IAV). In some embodiments, the nanoparticle increases the amount of IgA antibody in a subject.

In some aspects, disclosed herein is a method of reducing transmission of an influenza A virus (IAV) in a subject comprising administering to the subject a nanoparticle comprising chitosan and an inactivated influenza A virus (IAV) antigen, wherein the chitosan encapsulates the inactivated IAV antigen. In some embodiments, the method reduces nasal shedding of the IAV.

In some aspects, disclosed herein is a method of eliciting an immune response against swine influenza A virus in a subject comprising administering to the subject a nanoparticle comprising chitosan and an inactivated influenza A virus (IAV) antigen, wherein the chitosan encapsulates the inactivated IAV antigen. In some embodiments, the method elicits an increased amount of IgA antibody in the subject compared to a control.

### BRIEF DESCRIPTION OF THE DRAWINGS

Additional aspects and advantages of the disclosure will be set forth, in part, in the detailed description and any claims which follow, and in part will be derived from the detailed description or can be learned by practice of the various aspects of the disclosure. It is to be understood that both the foregoing general description and the following detailed description are exemplary and explanatory only and are not restrictive of the disclosure.

The accompanying drawings, which are incorporated in and constitute a part of this specification, illustrate certain examples of the present disclosure and together with the description, serve to explain, without limitation, the principles of the disclosure. Like numbers represent the same element(s) throughout the figures.

FIGS. 1A-1I. In vitro characteristics of CNPs-KAg. Diameter of (FIG. 1A) Empty chitosan nanoparticles (CNPs) and (FIG. 1B) SwIAV killed antigen (KAg) loaded CNPs (CNPs-KAg) determined by DLS. Scanning electron microscope (SEM) images of (FIG. 1C) Empty CNPs and (FIG. 1D) CNPs-KAg. (FIG. 1E) Release of KAg from CNPs-KAg suspended in PBS over a period of 15 days. (FIG. 1F) Uptake of soluble SwIAV KAg or CNPs-KAg formulation by monocytes/macrophages at indicated time points determined by fluorescent microscopy (Olympus, IX70, 20× magnifications). Frequency of monocytes/macrophages uptaken SwIAV KAg treated with soluble antigen or CNPs-KAg determined by flow cytometry: (FIG. 1G) SwIAV infected MDCK cells as positive control; (FIG. 1H) a representative picture of SwIAV KAg or CNPs-KAg uptake by porcine monocytes/macrophages after 150 min treatment; and (FIG. 1I) percentage of cells with internalized SwIAV antigen at 10 min, 30 min and 150 min treatment.

FIGS. 2A-2F. Production of innate, proinflammatory and Th1 cytokines by porcine MoDCs treated for 48 h with medium, KAg, CNPs-KAg or LPS control. Levels of cytokines (FIG. 2A) IFN-α; (FIG. 2B) TNF-α; (FIG. 2C) IL-1β; (FIG. 2D) IL-12; (FIG. 2E) IL-6; and (FIG. 2F) IL-10 were estimated in stimulated cell culture supernatant by ELISA. Data represents mean value of 7 pig derived DCs±SEM. Statistical analysis between two groups was carried out using Mann-Whitney test. Asterisk refers to a statistically significant difference between the indicated two pig groups (* p<0.05, ** p<0.01 and ***p<0.001).

FIGS. 3A-3I. Antibody response after CNPs-KAg prime-boost vaccination at day post-vaccination 35/day post-challenge 0 (DPV 35/DPC 0) in pigs. Mucosal secretory IgA antibody response in nasal swab, systemic IgG antibody and hemagglutination inhibition (HI) titers in serum samples against: (FIG. 3A, FIG. 3D, FIG. 3G) H1N2-OH10; (FIG. 3B, FIG. 3E, FIG. 3H) H1N1-OH7; and (FIG. 3C, FIG. 3F, FIG. 3I) H3N2-OH4 IAVs. Data represents mean value of 3 to 5 pigs ±SEM. Statistical analysis was carried out using Kruskal-Wallis test followed by Dunn's post-hoc test. Asterisk refers to a statistically significant difference between the indicated two pig groups (* p<0.05). In antibody dilution curves (FIGS. 3A-F); A, B and C refers to significant difference between unvaccinated vs KAg-vaccinates; unvaccinated vs CNPs-KAg-vaccinates; and KAg vs CNPs-KAg-vaccinates, respectively, at the indicated dilution.

FIGS. 4A-4B. Expression of Th1 and Th2 response inducing specific transcription factors after prime-boost vaccination in pigs. The expression of (FIG. 4A) Th2 transcription factor GATA-3 and (FIG. 4B) Th1 transcription factor T-bet in PBMCs of pigs at DPV 35/DPC 0 were determined by qRT-PCR. Data represents mean value of 3 to 4 pigs ±SEM. Statistical analysis was carried out using Kruskal-Wallis test followed by Dunn's post-hoc test. Asterisk refers to a statistically significant difference between the indicated two pig groups (* p<0.05).

FIGS. 5A-5I. Mucosal IgA antibody response in the respiratory tract of pigs vaccinated with CNPs-KAg at day post-challenge 6. Specific IgA antibody response in nasal swab, BAL fluid and lung lysate samples against H1N2-OH10 (FIG. 5A, FIG. 5D, FIG. 5G), H1N1-OH7 (FIG. 5B, FIG. 5E, FIG. 5H) and H3N2-OH4 (FIG. 5C, FIG. 5F, FIG. 5I) IAVs. Data represents mean value of 3 to 5 pigs ±SEM at all indicated dilutions. Statistical analysis was carried out using Kruskal-Wallis test followed by Dunn's post-hoc test where A, B and C refers to significant difference (p<0.05) between unvaccinated vs KAg-vaccinates; unvaccinated vs CNPs-KAg-vaccinates and KAg vs CNPs-KAg-vaccinates, respectively, at the indicated dilution.

FIGS. 6A-6F. Serum IgG response and BAL fluid HI antibody titers in pigs vaccinated with CNPs-KAg at day post-challenge 6. Specific IgG antibody response in serum and BAL fluid HI titers against H1N2-OH10 (FIG. 6A, FIG. 6D), H1N1-OH7 (FIG. 6B, FIG. 6E) and H3N2-OH4 (FIG. 6C, FIG. 6F) IAVs. Data represents mean value of 3 to 5 pigs ±SEM. Statistical analysis was carried out using Kruskal-Wallis test followed by Dunn's post-hoc test. Asterisk refers to a statistically significant difference between the indicated two pig groups (* p<0.05). In antibody dilution curves (A-C); A, B and C refers to significant difference between unvaccinated vs KAg-vaccinates; unvaccinated vs CNPs-KAg-vaccinates and KAg vs CNPs-KAg-vaccinates, respectively, at the indicated dilution.

FIGS. 7A-7G. Cell-mediated immune response after prime-boost vaccination was enhanced in CNPs-KAg vaccinated pigs at pre-challenge DPV 35/DPC 0. PBMCs isolated from blood were stimulated with different variant IAVs. IFNγ secretion in the culture supernatant and antigen specific lymphocyte proliferation was determined after 72 h of stimulation with (FIG. 7A, FIG. 7E) H1N2-OH10, (FIG. 7B, FIG. 7F) H1N1-OH7 and (FIG. 7C, FIG. 7G) H3N2-OH4 IAVs. (FIG. 7D) Flow cytometry analysis of PBMCs showed enhanced frequency of CTLs (CD3⁺CD4⁻CD8αβ⁺) in CNPs-KAg vaccinated pigs. Data represents mean value of 3 to 5 pigs ±SEM. Statistical analysis was carried out using Kruskal-Wallis test followed by Dunn's post-hoc test. Asterisk refers to a statistically significant difference between the indicated two pig groups (* p<0.05).

FIGS. 8A-8D. Clinical and pathological changes in pigs vaccinated with CNPs-KAg post-challenge. (FIG. 8A) Rectal temperature was recorded daily after challenge until the day of necropsy. (FIG. 8B) Gross pneumonic lesions in lungs determined at DPC 6. (FIG. 8C) Representative H&E-stained lung pictures showing bronchial exudates (dotted black circle), perivascular inflammation (black arrow), peribronchial inflammation (dashed black arrow) and interstitial pneumonia (small black triangle). (FIG. 8D) Secretion of cytokine IL-6 in BAL fluid. Data represents mean value of 3 to 5 pigs ±SEM.

FIGS. 9A-9F. Cell-mediated immune response in TBLN-MNCs of pigs vaccinated with CNPs-KAg at day post-challenge 6. TBLN-MNCs isolated on the day of necropsy were stimulated with different variant SwIAVs, and secreted IFNγ into the culture supernatant was measured by cytokine ELISA against: (FIG. 9A) H1N2-OH10; (FIG. 9B) H1N1-OH7; and (FIG. 9C) H3N2-OH4 IAVs. (FIG. 9D) The frequency of T helper/memory cells (CD3⁺CD4⁻CD8α⁺) in TBLN-MNCs of CNPs-KAg vaccinated pigs were analyzed by flow cytometry. Expression of Th1 (FIG. 9E) and Th2 (FIG. 9F) transcription factors were also determined in TBLN at DPC 6. Data represents mean value of 3 to 5 pigs ±SEM. Statistical analysis was carried out using Kruskal-Wallis test followed by Dunn's post-hoc test. Asterisk refers to a statistically significant difference between the indicated two pig groups (* p<0.05).

FIGS. 10A-10C. Infectious challenge SwIAV H1N1 virus titer in the respiratory tract of CNPs-KAg vaccinated and IAV challenged pigs. Titers of challenge SwIAV shedding through nostrils at (FIG. 10A) DPC 4 and (FIG. 10B) DPC 6; and in BAL fluid at DPC 6 (FIG. 10C) determined by using cell culture technique. Data represents mean value of 3 to 5 pigs ±SEM. Statistical analysis was carried out using Kruskal-Wallis test followed by Dunn's post-hoc test. Asterisk refers to a statistically significant difference between the indicated two pig groups (* p<0.05).

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the terms “including,” “containing,” and variations thereof and are open, non-limiting terms. Although the terms “comprising,” “including,” and “containing” have been used herein to describe various embodiments, the terms “consisting essentially of” and “consisting of” can be used in place of “comprising,” “including,” and “containing” to provide for more specific embodiments and are also disclosed.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular nanoparticle is disclosed and discussed and a number of modifications that can be made to the nanoparticle are discussed, specifically contemplated is each and every combination and permutation of the nanoparticle and the modifications that are possible unless specifically indicated to the contrary. Thus, if a class of nanoparticles A, B, and C are disclosed as well as a class of nanoparticles D, E, and F and an example of a combination nanoparticle, or, for example, a combination nanoparticle comprising A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

It is understood that the compositions disclosed herein have certain functions. Disclosed herein are certain structural requirements for performing the disclosed functions, and it is understood that there are a variety of structures which can perform the same function which are related to the disclosed structures, and that these structures will ultimately achieve the same result.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

As used in the specification and claims, the singular form “a,” “an,” and “the” include plural references unless the context clearly dictates otherwise. For example, the term “an agent” includes a plurality of agents, including mixtures thereof.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

An “immunogenic composition” is a composition of matter suitable for administration to a human or animal subject (e.g., in an experimental setting) that is capable of eliciting a specific immune response, e.g., against a pathogen, such as swine influenza A virus. As such, an immunogenic composition includes one or more antigens (for example, whole purified virus or antigenic subunits, e.g., polypeptides, thereof) or antigenic epitopes. An immunogenic composition can also include one or more additional components capable of eliciting or enhancing an immune response, such as an excipient, carrier, and/or adjuvant. In certain instances, immunogenic compositions are administered to elicit an immune response that protects the subject against symptoms or conditions induced by a pathogen. In some cases, symptoms or disease caused by a pathogen is prevented (or treated, e.g., reduced or ameliorated) by inhibiting replication of the pathogen following exposure of the subject to the pathogen. In the context of this disclosure, the term immunogenic composition will be understood to encompass compositions that are intended for administration to a subject or population of subjects for the purpose of eliciting a protective or palliative immune response against the virus (that is, vaccine compositions or vaccines).

An “antigen” is a compound, composition, or substance that can stimulate the production of antibodies and/or a T cell response in an animal, including compositions that are injected, absorbed or otherwise introduced into an animal. The term “antigen” includes all related antigenic epitopes. The term “epitope” or “antigenic determinant” refers to a site on an antigen to which B and/or T cells respond. The “dominant antigenic epitopes” or “dominant epitope” are those epitopes to which a functionally significant host immune response, e.g., an antibody response or a T-cell response, is made. Thus, with respect to a protective immune response against a pathogen, the dominant antigenic epitopes are those antigenic moieties that when recognized by the host immune system result in protection from disease caused by the pathogen. The term “T-cell epitope” refers to an epitope that when bound to an appropriate MEW molecule is specifically bound by a T cell (via a T cell receptor). A “B-cell epitope” is an epitope that is specifically bound by an antibody (or B cell receptor molecule). An antigen can also affect the innate immune response.

An “immune response” is a response of a cell of the immune system, such as, but not limited to, a B cell, T cell, or monocyte, to a stimulus. An immune response can be a B cell response, which results in the production of specific antibodies, such as antigen specific neutralizing antibodies. An immune response can also be a T cell response, such as a CD4+ T cell response or a CD8+ T cell response. In some cases, the response is specific for a particular antigen (that is, an “antigen-specific response”). An immune response can also include the innate response. If the antigen is derived from a pathogen, the antigen-specific response is a “pathogen-specific response.” A “protective immune response” is an immune response that inhibits a detrimental function or activity of a pathogen, reduces infection by a pathogen, or decreases symptoms (including death) that result from infection by the pathogen. A protective immune response can be measured, for example, by the inhibition of viral replication or plaque formation in a plaque reduction assay or ELISA-neutralization assay, or by measuring resistance to pathogen challenge in vivo.

The immunogenic compositions disclosed herein are suitable for preventing, ameliorating and/or treating disease caused by infection of a virus, particularly influenza A virus (IAV).

The abbreviation “KAg” stands for killed antigen and represents the killed or inactivated virus or portions of a virus. The inactivated antigen comprises one or more immunogenic viral proteins and therefore the inactivated antigen can be an inactivated whole virus or a portion of a virus that is inactivated.

The abbreviation “CNP-KAg” or “CNPs-KAg” stands for chitosan nanoparticle-killed antigen. This represents the chitosan nanoparticle having encapsulated inactivated influenza A virus antigen.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “prevent” refers to a treatment that forestalls or slows the onset of a disease or condition or reduced the severity of the disease or condition. Thus, if a treatment can treat a disease in a subject having symptoms of the disease, it can also prevent that disease in a subject who has yet to suffer some or all of the symptoms.

“Administration” to a subject includes any route of introducing or delivering to a subject an agent. Administration can be carried out by any suitable route, including oral, topical, intravenous, subcutaneous, transcutaneous, transdermal, intramuscular, intra joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

“Pharmaceutically acceptable” component can refer to a component that is not biologically or otherwise undesirable, e.g., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value“10” is disclosed, then “about 10” is also disclosed.

### Chitosan Nanoparticle Compositions

It is understood that the nanoparticles of the present disclosure can be used in combination with the various compositions, methods, products, kits, and applications disclosed herein.

Disclosed herein is a novel nanoparticle. The nanoparticle comprises chitosan, and further comprises an inactivated influenza A virus (IAV) antigen. The nanoparticle is configured such that the chitosan encapsulates the inactivated IAV antigen.

The herein disclosed nanoparticles have numerous advantageous properties, including but not limited to, capacity to stimulate desirable immune responses including increased secretion of mucosal IgA antibodies, to reduce viral transmission (e.g., via nasal shedding route), and to provide effective immunity to one or more homologous, heterologous, and heterosubtypic influenza A viruses (IAVs). The nanoparticles further are comprised of a biocompatible, biodegradable, mucoadhesive, polycationic, and immunomodulatory design, can deliver highly immunostimulatory antigens, and have desirable size and encapsulation efficiency.

The nanoparticle comprises chitosan. Chitosan is a polysaccharide containing linkages of acetylated and deacetylated glucosamine and is commercially available from numerous sources. Chitosan is derived from chitin, an N-acetylglucosamine polymer common in fungi, arthropods, and other organisms. Chitosan is often formed from alkaline treatment of chitin in crustacean shells. Chitosan is biocompatible, non-toxic, and biodegradable, and is thus advantageous for use as a polymer to form a nanoparticle for administration to a mammalian subject.

Chitosan is typically composed of β-(1-4)-linked d-glucosamine and N-acetyl-d-glucosamine randomly distributed within the polymer. The degree of deacetylation of glucosamine subunits can affect the properties of chitosan, e.g., immunogenic properties. The degree of deacetylation can be described by the molar fraction of deacetylated units or percentage of deacetylation, and the molecular weight of chitosan. See Cheung et. al., Mar Drugs. 2015 August; 13(8): 5156-5186. In some embodiments, the nanoparticles comprise chitosan that is at least 50% deacetylated. In some embodiments, the nanoparticles comprise chitosan that is at least 55%, at least 60%, at least 65%, at least 70%, at least 75%, at least 80%, at least 85%, at least 90%, at least 95%, at least 98%, or at least 99% deacetylated. In some embodiments, the chitosan has a molecular weight up to 500,000 Da, up to 375,000 Da, up to 300,000 Da, up to 250,000 Da, up to 220,000 Da, or up to 190,000 Da. In some embodiments, the chitosan has a molecular weight of at least 10,000 Da, at least 20,000 Da, at least 30,000 Da, at least 40,000 Da, or at least 50,000 Da. In some embodiments, the chitosan has a molecular weight ranging from about 1,000 Da to about 500,000 Da, from about 10,000 Da to about 375,000 Da, from about 20,000 Da to about 300,000 Da, from about 30,000 Da to about 250,000 Da, from about 40,000 Da to about 220,000 Da, or from about 50,000 Da to about 190,000 Da.

Chitosan can be used in combination with additional compounds and polymers suitable for forming a nanoparticle. For example, the chitosan can be combined with natural or synthetic polymers, lipids, saccharides and polysaccharides, proteins, ligands such as targeting molecules, and other molecules or polymers suitable for forming a nanoparticle within the spirit of the invention.

In some embodiments, the nanoparticle further comprises a crosslinking agent. The crosslinking agent can crosslink chitosan polymers to aid in formation of the nanoparticle. In some embodiments, the crosslinking agent is negatively charged. In some embodiments, the crosslinking agent comprises tartaric acid (e.g., L(+)-tartaric acid), a carboxy-modified polyethylene glycol (e.g., poly(ethylene glycol) bis(carboxymethyl) ether), an acetic acid derivative (e.g., 4-phenylenediacetic acid), an isophthalic acid derivative (e.g., 5-sulfoisophthalic acid), or tripolyphosphate (TPP).

In some embodiments, the crosslinking agent comprises TPP. Because TPP has low toxicity in vivo, the useable amounts of TPP are not particularly limited. The nanoparticle can comprise a chitosan:TPP ratio, on a weight per volume basis, up to about 100:1, up to about 50:1, up to about 25:1, up to about 10:1, up to about 5:1, up to about 4:1, up to about 3:1, up to about 2:1, up to about 1:1, up to about 0.9:1, up to about 0.8:1, up to about 0.7:1, up to about 0.6:1, up to about 0.5:1, up to about 0.1:1, up to about 0.05:1, or up to about 0.01:1. As a non-limiting example, the nanoparticle can comprise about 1% chitosan (w/v) and about 0.5% TPP (w/v), resulting in a chitosan:TPP ratio, on a weight per volume basis, of up to about 2:1.

In some embodiments, the empty or unloaded nanoparticle (nanoparticle which does not contain encapsulated inactivated IAV antigen) comprises at least 50% by weight chitosan. In some embodiments, the unloaded nanoparticle comprises at least 55%, at least 60%, at least 65%, at least 70%, at least 75%, at least 80%, at least 85%, at least 90%, at least 95%, or at least 99% chitosan. Percent chitosan can be determined by several measures, including percent by dry weight, percent by weight per volume, percent by molar ratio or molarity, or by other known measures.

The nanoparticle comprises an inactivated influenza A virus (IAV) antigen. Any suitably immunogenic inactivated swine influenza A virus or swine influenza A virus antigen can be used. For example, the swine influenza A virus antigen can be a swine influenza A virus surface glycoprotein. Examples of immunogenic antigens include recombinantly derived hemagglutinin, neuraminidase, nucleocapsid and matrix proteins. The swine influenza A virus antigen can be recombinantly derived. In some embodiments, a portion of the inactivated IAV antigen is derived from an IAV which is not a swine IAV (e.g., an avian IAV or human IAV). As non-limiting examples, the inactivated IAV antigen can comprise a swine hemagglutinin protein and an avian neuraminidase protein, or alternatively can comprise an inactivated whole swine IAV and an inactivated whole human IAV. The inactivated IAV antigen can be the entire inactivated virus or can be a portion of the inactivated virus (e.g., all or portions of hemagglutinin and/or neuraminidase proteins). In some embodiments, the inactivated IAV antigen is from an H1N1, H1N2 or H3N2 IAV; however, any suitable IAV can be used for preparation of the inactivated IAV antigen.

As used herein, an “inactivated” virus is a virus grown in culture which, by any number of suitable methods, is caused to lose the ability to replicate. Thus, an inactivated virus cannot replicate in vivo or in vitro. The term is intended to encompass a virus which is killed. In one embodiment, the swine influenza A virus is inactivated by UV light. Other means of inactivation include chemical, heat, or radioactivity.

The nanoparticles are typically mucoadhesive, meaning the nanoparticles have the capability to adhere to mucus or mucosal surfaces in a subject. Cationic chitosan is a mucoadhesive polymer due to its positive charges. Mucoadhesion is advantageous for mucosal drug delivery because it can prolong the residence time of the nanoparticles and increase the half-time of mucociliary clearance at epithelial surfaces.

In some embodiments, the nanoparticle has a positive zeta potential. In some embodiments, the nanoparticle has a net positive charge at neutral pH. In some embodiments, the nanoparticle comprises one or more amine moieties at its surface. In some embodiments, the nanoparticle comprises polymerized, positively charged glucosamine monomers at its surface.

The nanoparticle is configured such that the chitosan encapsulates the inactivated IAV antigen. By “encapsulated,” it is meant there is a physical encasing of the inactivated IAV antigen in the nanoparticle, such that access to the inactivated IAV antigen is generally blocked. In some embodiments, the nanoparticle comprising chitosan encapsulates the inactivated IAV antigen such that the composition can elicit an immune response. However, due to inefficiencies in the encapsulation process, some inactivated IAV antigen can be associated on the outer surface of the nanoparticle.

In some embodiments, the nanoparticle is formed by self-assembly. Self-assembly refers to the process of forming a nanoparticle using components that will orient themselves in a predictable manner, thereby forming nanoparticles predictably and reproducibly. In some embodiments, the nanoparticles are formed using amphiphilic biomaterials which orient themselves with respect to one another to form nanoparticles of predictable dimension, constituents, and placement of constituents. In some embodiments, the nanoparticle is formed by ionic gelation.

Nanoparticles can aid the delivery of the inactivated IAV antigen and/or can also be immunogenic. Delivery can be to a particular site of interest, e.g. the mucosa. In some embodiments, the nanoparticle can create a timed release of the inactivated IAV antigen to enhance and/or extend the immune response.

The nanoparticle can have a diameter within the nanometer range (e.g., from 1 nm to 1,000 nm). However, the diameter of the nanoparticle is not specifically limited to the nanometer range and, in some embodiments, can be larger than 1,000 nm (e.g., up to 1,500 nm, up to 2,000 nm, or up to 3,000 nm or more). In some embodiments, the nanoparticle has a diameter of 1,000 nm or less, 750 nm or less, 500 nm or less, 400 nm or less, 300 nm or less, or 200 nm or less. In some embodiments, the nanoparticle has a diameter from 10 nm to less than 1,000 nm. Optionally, the nanoparticle has a diameter from 100 nm to 900 nm, from 200 nm to 800 nm, from 300 nm to 700 nm, from 400 nm to 600 nm, or from 450 nm to 550 nm. In some, embodiments, the nanoparticle has a diameter of about 500 nm.

The encapsulation efficiency of the nanoparticle for inactivated IAV antigen can vary. Encapsulation efficiencies are generally calculable based on the amount of inactivated IAV antigen used in the encapsulation procedure and the amount of inactivated IAV antigen which remains in solution after the nanoparticles are removed from the encapsulation solution. The nanoparticle can have an encapsulation efficiency of 60% or greater of the inactivated IAV antigen. In some embodiments, the nanoparticle has an encapsulation efficiency of 65% or greater, 70% or greater, or 75% or greater of the inactivated IAV antigen. In some embodiments, the nanoparticle has an encapsulation efficiency of about 67% of the inactivated IAV antigen.

In some embodiments, the nanoparticle composition can include unencapsulated inactivated IAV antigen. For instance, the nanoparticle can in some embodiments further comprise inactivated IAV antigen associated on the outer surface of the nanoparticle. The nanoparticles can be administered as nanoparticles which comprise only encapsulated inactivated IAV antigen (e.g., further purified nanoparticles), as nanoparticles comprising encapsulated inactivated IAV antigen and unencapsulated but associated (e.g., nanoparticle outer surface-associated) inactivated IAV antigens, or mixtures thereof.

The nanoparticle can have properties expressed in comparison to a control. As used herein, a “control” refers to a composition which can be administered to a subject to provide comparative data. Alternatively, a control can be a collection of values used as a standard applied to one or more subjects (e.g., a general number or average that is known and not identified in the method using a sample). In some embodiments, the control comprises an empty nanoparticle (e.g., a nanoparticle comprising chitosan, but which contains no encapsulated or associated inactivated IAV antigen). In some embodiments, the control comprises a solution, culture medium, or carrier in which nanoparticles can be added (e.g., water, DMEM). In some embodiments, the control comprises unencapsulated inactivated IAV antigen (KAg), typically in combination with a solution, culture medium, or carrier. In some embodiments, the control can be a different nanoparticle comprising an inactivated IAV antigen (e.g., a PLGA-containing nanoparticle) or a vaccine comprising a nanoparticle comprising an inactivated IAV antigen.

The nanoparticle can be used to prevent or treat influenza A virus infections in a subject. Typically, influenza A virus infection (or exposure thereto) occurs subsequent to administering the nanoparticle to a subject. The nanoparticle can comprise an inactivated IAV antigen which is homologous, heterologous, or heterosubtypic to the influenza A virus causing infection. As used herein, an influenza A virus is “homologous” to the inactivated IAV antigen if the influenza A virus contains the same hemagglutinin (H) and neuraminidase (N) surface proteins as the H and N surface proteins of the virus from which the inactivated IAV antigen is derived (e.g., an inactivated IAV antigen from an H1N2 influenza A virus is homologous to an H1N2 influenza A virus). As used herein, an influenza A virus is “heterologous” to the inactivated IAV antigen if the influenza A virus contains the same hemagglutinin (H) surface protein but a different neuraminidase (N) surface protein as compared to the H and N surface proteins of the virus from which the inactivated IAV antigen is derived (e.g., an inactivated IAV antigen from an H1N2 influenza A virus is heterologous to an H1N1 influenza A virus). As used herein, an influenza A virus is “heterosubtypic” to the inactivated IAV antigen if the influenza A virus contains a different hemagglutinin (H) surface protein as compared to the H surface protein of the virus from which the inactivated IAV antigen is derived (e.g., an inactivated IAV antigen from an H1N2 influenza A virus is heterosubtypic to an H3N2 influenza A virus). As such, a heterosubtypic relationship can include the same or different neuraminidase (N) surface protein.

In some embodiments, the nanoparticle prevents or reduces the symptoms of infection by more than one IAV, each of which are separately homologous, heterologous, or heterosubtypic to the inactivated IAV antigen comprised in the nanoparticle. In some embodiments, the IAV causing infection comprises H1N1, H1N2, H2N2, H2N3, H3N1, H3N2, H3N8, H5N1, H5N2, H5N3, H5N6, H5N8, H5N9, H6N1, H7N1, H7N2, H7N3, H7N4, H7N7, H7N9, H9N2, or H10N7 IAV. In some embodiments, the IAV is a swine IAV. In some embodiments, the IAV comprises H1N1, H1N2, H3N1, H3N2, H2N3, H4N6, or H9N2 IAV. In some embodiments, the IAV comprises an H1N1, H1N2, or H3N2 IAV. However, in some embodiments, the nanoparticles elicit cross-reactive responses to a range of IAVs (e.g., more than one) and thus, the IAV, or combinations thereof, need not be particularly limited.

The nanoparticle can reduce transmission of an IAV. As used herein, an IAV (e.g., an IAV which is reduced in nasal shedding, or for which immune responses are generated against) is an infectious IAV, particularly a swine IAV. An infectious IAV has the capability of infecting a subject, particularly a pig, and to cause symptoms of infection therein. In some embodiments, the nanoparticle reduces nasal shedding of an IAV compared to a control. Viral nasal shedding is an important mode of transmission of IAVs. Reduced nasal shedding can be determined in a sample collected from the upper respiratory tract of a subject by any suitable method, for example by nasal swab of shedding mucous.

The nanoparticles can reduce the amount of IAV in the upper respiratory tract of a subject by an amount quantifiable by any suitable method. In some embodiments, the amount of reduction of IAV in the upper respiratory tract of a subject is quantifiable by the tissue culture infective dose (TCID₅₀/mL) method. See, e.g., Reed et al., Am. J. Hyg., 27(3):493-7 (1938). In some embodiments, the nanoparticle reduces the amount of the IAV in an upper respiratory tract by at least 1×10¹ TCID₅₀/mL compared to a control. In some embodiments, the nanoparticle reduces the amount of the IAV in an upper respiratory tract, as compared to a control, by at least 1×10^{1.2}, at least 1×10^{1.4}, at least 1×10^{1.5}, at least 1×10^{1.6}, at least 1×10^{1.8}, at least 1×10^{2.0}, at least 1×10^{2.5}, at least 1×10^{3.0}, at least 1×10^{3.5}, at least 1×10^{4.0}, at least 1×10^{4.5}, or at least 1×10^{5.0} TCID₅₀/mL, or more.

The reduction in the amount of the IAV in the upper respiratory tract of a subject administered with the nanoparticles can occur shortly after exposure to, or infection with, the IAV. In some embodiments, the amount of IAV in the upper respiratory tract of a subject can be reduced within one, two, three, four, five, six, or seven days of exposure to the IAV. In a particular embodiment, the nanoparticle reduces the amount of IAV in the upper respiratory tract of a subject within four days of exposure to the IAV. In some or further embodiments, the nanoparticle further reduces the amount of IAV in the upper respiratory tract within six days of exposure to the IAV. In some embodiments, the IAV in a subject administered with the nanoparticles is reduced to below clinical detection levels at least one, two, three, four, five, six, seven, or more days earlier than in a subject administered with a control. In some embodiments, the nanoparticle prevents an increase in the amount of infecting IAV to a level which causes symptoms of the infection (e.g., flu-like symptoms).

In some embodiments, the nanoparticle reduces the amount of IAV in the lower respiratory tract, compared to a control. A sample can be collected from the lower respiratory tract of a subject by any suitable method, for example by bronchoalveolar lavage (BAL) of the lungs to obtain BAL fluid. In some embodiments, the reduced amount of an IAV in the lower respiratory tract can be in addition to reduced nasal shedding of the IAV.

The amount of reduction of IAV in the lower respiratory tract of a subject administered with the nanoparticles can be quantified by any suitable method (e.g., by TCID₅₀/mL). In some embodiments, the nanoparticle reduces the amount of the IAV in the lower respiratory tract (e.g., BAL fluid) of a subject by at least 1×10¹ TCID₅₀/mL compared to a control. In some embodiments, the nanoparticle reduces the amount of the IAV in the lower respiratory tract of a subject, as compared to a control, by at least 1×10^{1.2}, at least 1×10^{1.4}, at least 1×10^{1.5}, at least 1×10^{1.6}, at least 1×10^{1.8}, at least 1×10^{2.0}, at least 1×10^{2.5}, at least 1×10^{3.0}, at least 1×10^{3.5}, at least 1×10^{4.0}, at least 1×10^{4.5}, or at least 1×10^{5.0} TCID₅₀/mL.

The reduction in the amount of the IAV in the lower respiratory tract of a subject administered with the nanoparticles can occur shortly after exposure to, or infection with, the IAV. In some embodiments, the nanoparticle reduces the amount of IAV in the lower respiratory tract (e.g., BAL fluid) of a subject within one, two, three, four, five, six, or seven days of exposure to the IAV. In a particular embodiment, the nanoparticle reduces the amount of IAV in the lower respiratory tract within four days of exposure to the IAV. In some or further embodiments, the nanoparticle further reduces the amount of IAV in the lower respiratory tract within six days of exposure to the IAV. In some embodiments, the amount of IAV in the lower respiratory tract of a subject administered with the nanoparticles is reduced to below clinical detection levels at least one, two, three, four, five, six, seven, or more days earlier than in a subject administered with a control.

The nanoparticles disclosed herein can boost humoral immune responses. For example, the nanoparticles can boost levels of one or more types of antibodies such as, and without limitation, IgA and IgG antibodies. In some embodiments, the nanoparticle elicits an increased amount of IgA antibody in a subject compared to a control. Increased IgA antibody can be determined in relevant samples from a subject, for example serum, mucosa, and/or secretory fluids such as saliva. In a particular embodiment, the nanoparticle increases the amount of IgA antibody in the respiratory mucosa. Increased IgA antibodies can be elicited in any one or more areas of the respiratory mucosa comprising, for example, the upper respiratory tract (determined in, for example, nasal swab), lower respiratory tract (determined in, for example, BAL fluid), lung parenchyma (determined in, for example, lung lysates), or combinations thereof, or in other areas of the respiratory mucosa. In such an embodiment, increased mucosal IgA can facilitate reduced nasal shedding of an IAV and/or enhance clearance of an IAV in a subject.

In some embodiments, the nanoparticle elicits at least 20% more IgA antibody in a subject compared to a control. In some embodiments, the nanoparticle elicits at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, or at least 200% or more IgA antibody in a subject compared to a control.

In some embodiments, the nanoparticle elicits an increased amount of IgG antibody in a subject compared to a control. Increased IgG antibody can be determined in relevant samples from a subject, for example blood, plasma, serum, or lymph.

In some embodiments, the nanoparticle elicits at least 20% more IgG antibody in a subject compared to a control. In some embodiments, the nanoparticle elicits at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, or at least 200% or more IgG antibody in a subject compared to a control.

The subject can be any mammalian subject, for example a human, pig, dog, cow, horse, mouse, rabbit, etc. In some embodiments, the subject is a livestock mammal, particularly a pig. In some embodiments, the subject is a pig at elevated risk for IVA infection, for example by housing in quarters in close proximity to avian livestock (e.g., chickens) which can be or are infected with an avian IVA, or in close proximity to a human which can be or is infected with a human IVA. The subject can be a male or female of any age, size, or other general classifiers.

Optionally, the nanoparticle can be formulated in a medicament. The nanoparticle can be formulated in any suitable medicament including, for example, but not limited to, solids, semi-solids, liquids, and gaseous (inhalant) dosage forms, such as tablets, pills, powders, liquid solutions or suspensions, suppositories, injectables, infusions, inhalants, hydrogels, topical gels, sprays, and the like. Optionally, the medicament comprises a pharmaceutically acceptable excipient/carrier. Optionally, the medicament comprises an effective dose of the inactivated IAV antigen (e.g., a dose effective to prevent or reduce symptoms of an IAV infection).

Also described herein are vaccines comprising an immunogenic composition disclosed herein in an immunogenic carrier, wherein the vaccine is protective against IAV, particularly swine IAV infection. In some embodiments, the vaccine further comprises a pharmaceutically acceptable carrier. The term “immunogenic carrier” as used herein can refer to a first polypeptide or fragment, variant, or derivative thereof which enhances the immunogenicity of a second polypeptide or fragment, variant, or derivative thereof. An “immunogenic carrier” can be fused, to or conjugated/coupled to the desired polypeptide or fragment thereof, or provided therewith. See, e.g., European Patent No. EP 0385610 B1, which is incorporated herein by reference in its entirety. An example of an “immunogenic carrier” is chitosan. In some embodiments the vaccine can comprise chitosan, inactivated swine IAV antigen, and a pharmaceutically acceptable carrier, wherein the chitosan encapsulates inactivated swine IAV antigen.

Disclosed are illustrative immunogenic compositions, e.g., vaccine compositions. Additionally, the compositions described herein can comprise one or more immunostimulants. An immunostimulant refers to essentially any substance that enhances or potentiates an immune response (antibody or cell-mediated) to an exogenous antigen. One preferred type of immunostimulant comprises an adjuvant. Many adjuvants contain a substance designed to protect the antigen from rapid catabolism, such as aluminum hydroxide or mineral oil, and a stimulator of immune responses, such as lipid A, Bordetella pertussis or Mycobacterium tuberculosis derived proteins. Certain adjuvants are commercially available as, for example, Freund's Incomplete Adjuvant and Complete Adjuvant (Difco Laboratories, Detroit, Mich.); Merck Adjuvant 65 (Merck and Company, Rahway, N.J.); AS-2 (GlaxoSmithKline, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF, interleukin-2, -7, -12, and other like growth factors, may also be used as adjuvants.

The adjuvant can induce an anti-inflammatory immune response (antibody or cell-mediated). Accordingly, high levels of anti-inflammatory cytokines (anti-inflammatory cytokines may include, but are not limited to, interleukin 4 (IL-4), interleukin 5 (IL-5), interleukin 10 (IL-10), and transforming growth factor beta (TGFβ). Optionally, an anti-inflammatory response can be mediated by CD4+ T helper cells. Bacterial flagellin has been shown to have adjuvant activity (McSorley et al., J. Immunol. 169:3914-19, 2002). Also disclosed are polypeptide sequences that encode flagellin proteins that can be used in adjuvant compositions. Additional adjuvants include but are not limited to, monophosphoryl lipid A (MPL), aminoalkyl glucosaminide 4-phosphates (AGPs), including, but not limited to RC-512, RC-522, RC-527, RC-529, RC-544, and RC-560 (Corixa, Hamilton, Mont.).

In addition, the adjuvant can be one that induces an immune response predominantly of the Th1 type. High levels of Th1-type cytokines (e.g., IFN-γ, TNFα, IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. In some embodiments, particularly those in which increased secretion of IgA and IgG antibodies are desirable, the adjuvant can be on in which induces an immune response predominately of the Th2-type. Following application of a vaccine as provided herein, a subject will support an immune response that includes Th1- and Th2-type responses. Optionally, the level of Th1-type cytokines will increase to a greater extent than the level of Th2-type cytokines. The levels of these cytokines may be readily assessed using standard assays. The level of Th2-type cytokines can optionally increase to a greater extent than the level of Th1-type cytokines.

Additional illustrative adjuvants for use in the disclosed compositions (e.g., vaccines) include, for example, a combination of monophosphoryl lipid A, preferably 3-de-O-acylated monophosphoryl lipid A, together with an aluminum salt adjuvants available from Corixa Corporation (Seattle, Wash.; see, for example, U.S. Pat. Nos. 4,436,727; 4,877,611; 4,866,034 and 4,912,094); CpG-containing oligonucleotides (in which the CpG dinucleotide is unmethylated; see e.g., WO 96/02555, WO 99/33488 and U.S. Pat. Nos. 6,008,200 and 5,856,462); immunostimulatory DNA sequences (see e.g., Sato et al., Science 273:352, 1996); saponins such as Quil A, or derivatives thereof, including QS21 and QS7 (Aquila Biopharmaceuticals Inc., Framingham, Mass.); Escin; Digitonin; or Gypsophila or Chenopodium quinoa saponins; Montamide ISA 720 (Seppic, France), SAF (Chiron, Calif., United States), ISCOMS (CSL), MF-59 (Chiron), the SBAS series of adjuvants (e.g., SBAS-2 or SBAS-4, available from GlaxoSmithKline, Philadelphia, Pa.), Detox (Enhanzyn™) (Corixa, Hamilton, Mont.), RC-529 (Corixa, Hamilton, Mont.) and other aminoalkyl glucosaminide 4-phosphates (AGPs), such as those described in pending U.S. patent application Ser. Nos. 08/853,826 and 09/074,720; polyoxyethylene ether adjuvants such as those described in WO 99/52549A1; and combinations thereof. In some embodiments, the adjuvant comprises alpha Galactosylceramide.

In some embodiments, the comparison to a control is statistically significant, as shown by any suitable statistical method.

### Methods of Use

Also disclosed herein are methods of reducing transmission of an influenza A virus (IAV) in a subject compared to a control comprising administering to the subject a nanoparticle comprising chitosan and an inactivated influenza A virus (IAV) antigen, wherein the chitosan encapsulates the inactivated IAV antigen.

Also disclosed herein are methods of eliciting an immune response against swine influenza A virus in a subject comprising administering to the subject a nanoparticle comprising chitosan and an inactivated influenza A virus (IAV) antigen, wherein the chitosan encapsulates the inactivated IAV antigen.

Also disclosed herein are methods of preventing influenza A virus (IAV) infection in a subject compared to a control comprising administering to the subject a nanoparticle comprising chitosan and an inactivated influenza A virus (IAV) antigen, wherein the chitosan encapsulates the inactivated IAV antigen.

Also disclosed herein are methods of preventing symptoms of an influenza A virus (IAV) infection in a subject compared to a control comprising administering to the subject a nanoparticle comprising chitosan and an inactivated influenza A virus (IAV) antigen, wherein the chitosan encapsulates the inactivated IAV antigen.

In any of the methods disclosed herein, the nanoparticle can be any herein disclosed nanoparticle. Similarly, the subject can be any herein disclosed subject, particularly a pig.

In some embodiments, the administering step can include any method of introducing the particle into the subject appropriate for the nanoparticle formulation. In some embodiments, the nanoparticle is administered intranasally. In some embodiments, the nanoparticle is administered in a mist formulation in the nostril of a subject. In some embodiments, at least 1×10⁵ TCID₅₀/mL, at least 1×10⁶ TCID₅₀/mL, at least 1×10⁷ TCID₅₀/mL, or more nanoparticles are administered to the subject. In some embodiments, the administering step includes administering a vaccine comprising any herein disclosed nanoparticle and a pharmaceutically acceptable carrier.

The administering step can include at least one, two, three, four, five, six, seven, eight, nine, or at least ten dosages. The administering step can be performed before the subject exhibits disease symptoms (e.g., vaccination), or as disease symptoms occur. The administering step can be performed prior to, concurrent with, or subsequent to administration of other agents (e.g., adjuvants) to the subject. The administering step can be performed with or without co-administration of additional agents (e.g., immunostimulants).

The nanoparticle can comprise an inactivated IAV antigen which is homologous, heterologous, or heterosubtypic to the IAV of the exposure/infection. In some embodiments, the nanoparticle reduces transmission of, elicits an immune response against, prevents infection of, and/or prevents symptoms of an infection with IAV which is homologous, heterologous, or heterosubtypic to the inactivated IAV antigen comprised in the nanoparticle.

The IAV causing infection can comprise H1N1, H1N2, H2N2, H2N3, H3N1, H3N2, H3N8, H5N1, H5N2, H5N3, H5N6, H5N8, H5N9, H6N1, H7N1, H7N2, H7N3, H7N4, H7N7, H7N9, H9N2, or H10N7 IAV. In some embodiments, the IAV comprises a swine IAV. In some embodiments, the IAV comprises H1N1, H1N2, H3N1, H3N2, H2N3, H4N6, or H9N2 IAV. In some embodiments, the IAV comprises an H1N1, H1N2, or H3N2 IAV. In some embodiments, the subject is infected with more than one IAV, each of which are separately homologous, heterologous, or heterosubtypic to the inactivated IAV antigen comprised in the nanoparticle.

As is typical of many vaccines and prophylactic treatments, the duration of time between the administration step and a subsequent exposure/infection can occasionally affect the outcome of infection in the subject. In some embodiments, the administering step is performed at least three days, at least five days, at least one week, at least two weeks, at least three weeks, or at least one month prior to exposure of the subject to an IAV. In some embodiments, the administering step is performed at about 35 days prior to exposure of the subject to an IAV.

In some embodiments, the methods can comprise a second administration step (e.g., a vaccine boost). A second administration step can, in some embodiments, improve the subject's immune responses and clinical outcomes upon exposure to an IAV. In some embodiments, the second administration step can be performed at least three days, at least five days, at least one week, at least two weeks, at least three weeks, or at least one month after performing the first administration step.

In some embodiments, the methods can comprise a third, fourth, or fifth, etc. administration step. The nanoparticle of the second or any subsequent administration step can be the same nanoparticle as that of the first administration step, or alternatively, a different herein disclosed nanoparticle.

In some embodiments, the methods reduce nasal shedding of an IAV. The IAV is an infectious IAV and, in some further embodiments, a swine IAV. A sample can be collected from the upper respiratory tract of a subject by any suitable method (e.g., by nasal swab of shedding mucous).

The amount of reduction of IAV in the upper respiratory tract of a subject administered with the nanoparticles can be quantified by any suitable method. In some embodiments, the amount of reduction of IAV in the upper respiratory tract of a subject is quantifiable by TCID₅₀/mL. In some embodiments, the methods reduce the amount of the IAV in an upper respiratory tract of a subject by at least 1×10¹ TCID₅₀/mL compared to a control. In some embodiments, the methods reduce the amount of the IAV in an upper respiratory tract of a subject, as compared to a control, by at least 1×10^{1.2}, at least 1×10^{1.4}, at least 1×10^{1.5}, at least 1×10^{1.6}, at least 1×10^{1.8}, at least 1×10^{2.0}, at least 1×10^{2.5}, at least 1×10^{3.0}, at least 1×10^{3.5}, at least 1×10^{4.0}, at least 1×10^{4.5}, or at least 1×10^{5.0} TCID₅₀/mL.

In some embodiments, the methods reduce the amount of the IAV in the upper respiratory tract of a subject shortly after exposure to, or infection with, the IAV. In some embodiments, the methods reduce the amount of IAV in the upper respiratory tract of a subject within one, two, three, four, five, six, or seven days of exposure to the IAV. In a particular embodiment, the methods reduce the amount of IAV in the upper respiratory tract within four days of exposure to the IAV. In some or further embodiments, the methods further reduce the amount of IAV in the upper respiratory tract within six days of exposure to the IAV. In some embodiments, the methods reduce the levels of IAV in a subject to below clinical detection levels at least one, two, three, four, five, six, seven, or more days earlier than in a subject administered with a control. In some embodiments, the methods prevent an increase in the amount of infecting IAV to a level which causes symptoms of the infection (e.g., flu-like symptoms).

In some embodiments, the methods reduce the amount of IAV in the lower respiratory tract. Reduced IAV levels can be determined in a sample collected from the lower respiratory tract of a subject by any suitable method (e.g., in BAL fluid). In some embodiments, the reduced amount of an IAV in the lower respiratory tract can be in addition to reduced nasal shedding of the IAV.

The amount of reduction of IAV in the lower respiratory tract of a subject administered with the nanoparticles can be quantified by any suitable method (e.g., by TCID₅₀/mL). In some embodiments, the methods reduce the amount of the IAV in the lower respiratory tract (e.g., BAL fluid) of a subject by at least 1×10¹ TCID₅₀/mL compared to a control. In some embodiments, the methods reduce the amount of the IAV in an upper respiratory tract of a subject, as compared to a control, by at least 1×10^{1.2}, at least 1×10^{1.4}, at least 1×10^{1.5} at least 1×10^{1.6}, at least 1×10^{1.8}, at least 1×10^{2.0}, at least 1×10^{2.5}, at least 1×10^{3.0}, at least 1×10^{3.5}, at least 1×10^{4.0}, at least 1×10^{4.5}, or at least 1×10^{5.0} TCID₅₀/mL.

In some embodiments, the methods reduce the amount of the IAV in the lower respiratory tract of a subject administered with the nanoparticles shortly after exposure to, or infection with, the IAV. In some embodiments, the methods reduce the amount of IAV in the lower respiratory tract (e.g., BAL fluid) of a subject within one, two, three, four, five, six, or seven days of exposure to the IAV. In a particular embodiment, the methods reduce the amount of IAV in the lower respiratory tract of a subject within four days of exposure to the IAV and, in some or further embodiments, reduce the amount of IAV within six days of exposure to the IAV. In some embodiments, the methods reduce the amount of IAV in the lower respiratory tract to below clinical detection levels at least one, two, three, four, five, six, seven, or more days earlier than in a subject administered with a control.

The methods disclosed herein can boost humoral immune responses. For example, the methods can boost levels of one or more types of antibodies such as, and without limitation, IgA and IgG antibodies. In some embodiments, the methods increase the amount of IgA antibody in a subject. Increased IgA antibody can be determined in relevant samples from a subject, for example serum, mucosa, and/or secretory fluids such as saliva. In a particular embodiment, the methods elicit an increased amount of IgA antibody, as compared to a control, in the respiratory mucosa. For example, the methods increase IgA antibodies in any one or more areas of the respiratory mucosa comprising the upper respiratory tract (determined in, for example, nasal swab), lower respiratory tract (determined in, for example, BAL fluid), lung parenchyma (determined in, for example, lung lysates), or combinations thereof, or in other areas of the respiratory mucosa. In such embodiments, increased mucosal IgA can facilitate reduced nasal shedding of an IAV and/or enhance clearance of an IAV in a subject.

In some embodiments, the methods elicit at least 20% more IgA antibody in a subject. In some embodiments, the methods elicit at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, or at least 200% or more IgA antibody in a subject compared to a control.

In some embodiments, the methods elicit an increased amount of IgG antibody in a subject. Increased IgG antibody can be determined in relevant samples from a subject, for example blood, plasma, serum, or lymph. In some embodiments, the methods elicit at least 20% more IgG antibody in a subject compared to a control. In some embodiments, the methods elicit at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, or at least 200% or more IgG antibody in a subject compared to a control.

In some embodiments, the methods enhance cell-mediated immune responses to an IAV in a subject. In some embodiments, the methods elicit increased amounts of IFNγ in a subject compared to a control. Increased amounts of IFNγ can be determined in relevant samples from a subject, for example blood, plasma, serum, or lymph. In some embodiments, the methods elicit at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, or more IFNγ in a subject compared to a control. In some embodiments, the methods elicit at least 2-fold, at least 3-fold, at least 4-fold, at least 5-fold, at least 10-fold, at least 15-fold, at least 20-fold, or more IFNγ in a subject compared to a control. In some embodiments, the methods elicit increased amounts of IFNγ in a subject after administering the nanoparticles but before exposure to an IAV. In some or further embodiments, the methods elicit increased amounts of IFNγ in a subject after administering the nanoparticles and after exposure to an IAV.

In some embodiments, the methods can elicit enhanced helper T cell and memory T cell frequencies in a subject. Enhanced helper T cell and memory T cell frequency can be determined in relevant samples from a subject, for example blood, plasma, serum, or lymph. In some embodiments, the helper and memory T cells having enhanced frequency comprise CD3⁺CD4⁺CD8α⁺ cells. In some embodiments, the methods elicit at least 5%, at least 10%, at least 15%, or at least 20% more helper and memory T cells as a percentage of CD3⁺ cells in a subject compared to a control. In some embodiments, the methods elicit enhanced helper and memory T cell frequencies after exposure to an IAV.

In some embodiments, the methods elicit enhanced cytotoxic T cell (CTL) frequencies in a subject. Enhanced CTL frequency can be determined in relevant samples from a subject, for example blood, plasma, serum, or lymph. In some embodiments, the CTLs having enhanced frequency comprise CD3⁺CD4⁻CD8αβ⁺ cells. In some embodiments, the methods elicit at least 5%, at least 10%, at least 15%, or at least 20% more CTL as a percentage of CD3⁺ cells in a subject compared to a control.

In some embodiments, the methods reduce tissue damage in the lungs of a subject caused by subsequent IAV infection. The damage can be, for example, pneumonic lesions, microscopic lesions, or other IAV-induced tissue damage in the lungs. Reduced damage can be measured and demonstrated by, for example and without limitation, reduced percentage of lung consolidation, or by reduced interstitial pneumonia in lung tissue (e.g., in H&E stained tissue).

In some embodiments, the methods can reduce levels of pro-inflammatory cytokines in a subject. Reduced levels of pro-inflammatory cytokines can, in some embodiments, occur systemically (e.g., measured in serum), or can be measured locally in BAL fluid. In some embodiments, the pro-inflammatory cytokine comprises IL-6.

In some embodiments, the methods further comprise administering any one or more of the herein disclosed immunostimulants (e.g., adjuvants). The immunostimulant can be administered prior to, concurrent with, or subsequent to administration of the nanoparticles. In some embodiments, the nanoparticle can be provided in the form of a medicament or vaccine. The medicament or vaccine can further comprise a pharmaceutically acceptable excipient/carrier.

In some embodiments, the comparison to a control in any method is statistically significant, as shown by any suitable statistical method.

### Kits

Also disclosed herein are kits comprising a nanoparticle comprising chitosan and an inactivated influenza A virus (IAV) antigen, wherein the chitosan encapsulates the inactivated IAV antigen, and a device for intranasal administration.

In any of the kits disclosed herein, the nanoparticle can be any herein disclosed nanoparticle. Similarly, the kit can be used to administer the nanoparticle to any herein disclosed subject, particularly a pig.

The kit comprises a device for intranasal administration. The device for intranasal administration, in some embodiments, is configured for insertion into the nostril of a subject.

In some embodiments, the device provides a mist of liquid formulation comprising the nanoparticle. In some embodiments, the device provides a mist of a vaccine comprising the nanoparticle and a pharmaceutically acceptable carrier. In some embodiments, the device is a nasal spray device.

### EXAMPLES

To further illustrate the principles of the present disclosure, the following examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. These examples are not intended to exclude equivalents and variations of the present invention which are apparent to one skilled in the art. Unless indicated otherwise, temperature is ° C. or is at ambient temperature, and pressure is at or near atmospheric. There are numerous variations and combinations of process conditions that can be used to optimize product quality and performance. Only reasonable and routine experimentation will be required to optimize such process conditions.

### Example 1. Mucosal Immunity and Protective Efficacy of Intranasal Inactivated Influenza Vaccine is Improved by Chitosan Nanoparticle Delivery in Pigs

Annually, swine influenza A virus (SwIAV) causes severe economic loss to swine industry. Currently used inactivated SwIAV vaccines administered by intramuscular injection provide homologous protection, but limited heterologous protection against constantly evolving field viruses, attributable to induction of inadequate levels of mucosal IgA and cellular immune responses in the respiratory tract.

In this study, the immune responses and cross-protective efficacy of intranasally-delivered chitosan encapsulated inactivated SwIAV vaccine were evaluated in pigs. Killed SwIAV H1N2 (δ-lineage) antigens (KAg) were encapsulated in chitosan polymer-based nanoparticles (CNPs-KAg). The vaccine was administered twice intranasally as mist to nursery pigs. Vaccinates and controls were then challenged with a zoonotic and virulent heterologous SwIAV H1N1 (γ-lineage). Pigs vaccinated with CNPs-KAg exhibited enhanced IgG serum antibody and mucosal secretory IgA antibody responses in nasal swabs, bronchoalveolar lavage (BAL) fluids and lung lysates that were reactive against homologous (H1N2), heterologous (H1N1) and heterosubtypic (H3N2) IAV strains. Prior to challenge, increased frequency of cytotoxic T lymphocytes, antigen-specific lymphocyte proliferation and recall IFN-γ secretion by restimulated peripheral blood mononuclear cells in CNPs-KAg compared to control KAg-vaccinates were observed. In CNPs-KAg vaccinated pigs challenged with heterologous virus reduced severity of macroscopic and microscopic influenza-associated pulmonary lesions were observed. Importantly, the infectious SwIAV titers in nasal swabs (days post-challenge 4) and BAL fluid (days post-challenge 6) were significantly (p<0.05) reduced in CNPs-KAg vaccinates but not in KAg vaccinates when compared to the unvaccinated challenge controls. In addition, increased frequency of T-helper memory cells and increased levels of recall IFNγ secretion by tracheobronchial lymph nodes cells were observed.

In summary, chitosan SwIAV nanovaccine delivered by intranasal route elicited strong cross-reactive mucosal IgA and cellular immune responses in the respiratory tract that resulted in reduced nasal viral shedding and lung virus titers in pigs. Thus, chitosan-based influenza nanovaccine provides a superior vaccine for use in pigs, and pigs are a useful animal model for preclinical testing of particulate intranasal human influenza vaccines.

### Background

Influenza is caused by influenza A virus (IAV) of Orthomyxoviridae family. It is an economically important disease in the global pig industry (Dykhuis et al., 2012; Crisci et al., 2013). Virulent swine IAV (SwIAV) infection leads to acute febrile respiratory disease which is often complicated with secondary bacterial infections (Janke, 2013). SwIAV increases its genetic diversity through frequent antigenic drift and antigenic shift. So far H1N1, H1N2 and H3N2 subtypes are the major SwIAV circulating in pig populations (Vincent et al., 2008). Since epithelial cells lining the porcine respiratory tract bear receptors for both avian and human IAVs, pigs can be infected with IAV from different hosts and this event favors genetic assortment and adaptation of novel influenza strains of zoonotic and even pandemic potential (Ito et al., 1998). The pandemic H1N1 virus of 2009 and more recent ‘H3N2 variant’ virus in the US are recent examples of swine-origin IAVs which cause infection and resultant pulmonary disease in humans (Vincent et al., 2014; Schicker et al., 2016). Controlling influenza in pigs through vaccination serves dual benefits by protecting economic loss in swine industry and preventing possible public health risk that these reassorted SwIAVs pose for humans.

Swine influenza vaccines are commercially available. These are multivalent whole inactivated virus (WIV) vaccines that are administered intramuscularly (IM) (Vincent et al., 2017). The WIV vaccines provide protection against homologous virus infections but do not induce adequate heterologous immunity against constantly evolving IAVs that develop by point mutation(s) (Van Reeth and Ma, 2013; Vincent et al., 2017). Moreover, the IM route used for WIV vaccines does not elicit adequate mucosal immune responses which are essential for providing cross-protective immunity against multitude of variant IAVs (Tamura and Kurata, 2004; van Riet et al., 2012). Intranasal (IN) vaccine that targets mucosal immune system of the respiratory tract can be a useful alternative to the current IM influenza vaccines used in pigs. Nasal mucosal vaccination not only induces strong protective immune responses at mucosal sites in the respiratory tract but also enhances immunity at distal mucosal and systemic sites (Neutra and Kozlowski, 2006; Kim and Jang, 2017).

Biodegradable and biocompatible polymer-based nanoparticle (NP) formulation(s) provide an innovative strategy of vaccine antigen delivery to mucosal sites (Mishra et al., 2010). Particulate vaccines facilitate antigen uptake by professional antigen presenting cells (APCs), maintain slow and sustained antigen release, prevent the antigen(s) from undesirable enzymatic degradation and potentiate the levels of protective immunity (Mishra et al., 2010; Mahapatro and Singh, 2011). Different types of nanoparticles are investigated for IN delivery of influenza vaccine antigens. For example, IN immunization in mice using liposome based DNA and subunit influenza nanovaccines are shown to elicit mucosal, cellular and humoral immune responses (Babai et al., 1999; Wang et al., 2004). Poly(lactic-co-glycolic) acid (PLGA) nanoparticle entrapped highly conserved H1N1 influenza virus peptides administered IN enhances the epitope specific T cell response and protective efficacy in pigs (Hiremath et al., 2016). Ferritin based IN influenza nanovaccine is shown to enhance mucosal secretary IgA and T cell response and confers homo- and hetero-subtypic protection in mice (Qi et al., 2018). In a previous study, killed SwIAV antigen (KAg) encapsulated in PLGA polymer-based NP and delivered IN induced robust cross-reactive cell-mediated immune response associated with significant clearance of challenge heterologous virus from the lungs of pigs (Dhakal et al., 2017b). In another study, encapsulation of KAg in polyanhydride polymer-based NP also enhanced the cross-reactive cell-mediated immune response against SwIAV (Dhakal et al., 2017a). However, both PLGA and polyanhydride polymer-based NP SwIAV vaccines used IN in these studies failed to elicit mucosal IgA and systemic IgG antibody responses, most likely due to their biased ability to induce strong T helper 1 (Th1) but not Th2 responses. This Th1-biased response failed to reduce the nasal virus shedding in pigs (Dhakal et al., 2017a; Dhakal et al., 2017b).

In the present example, chitosan, a natural mucoadhesive polymer, was used for encapsulation of SwIAV KAg (CNPs-KAg) and a heterologous vaccine-challenge trial in nursery pigs was performed. Due to its cationic nature, chitosan binds readily to mucosal surfaces. Chitosan also possesses adjuvant properties, a feature which promotes immune activation (van der Lubben et al., 2001). Previous studies have shown that chitosan nanoparticles form an attractive platform for mucosal vaccine delivery. For example, live Newcastle disease virus (NDV) encapsulated in chitosan nanoparticle and delivered through oral and intranasal route in chickens induced higher secretary IgA antibody response in intestinal mucosa and enhanced protective efficacy against highly virulent NDV strain challenge infection (Zhao et al., 2012). Similarly, influenza subunit/split virus vaccine delivered in chitosan nanoparticle by IN route improves systemic and mucosal antibody and cell-mediated immune responses in mice (Amidi et al., 2007; Sawaengsak et al., 2014; Liu et al., 2015). The results here demonstrated that CNPs-KAg IN vaccination improved mucosal IgA response in the entire respiratory tract and also elicited cell-mediated immune response against different subtypes of SwIAV resulting in reduced nasal viral shedding and infectious virus burden in the pulmonary parenchyma.

### Materials and Methods

SwIAV Propagation and Inactivation

Field isolates of IAVs A/Swine/OH/FAH10-1/10 (H1N2) (Ali et al., 2012), A/Swine/OH/24366/2007 (H1N1) (Yassine et al., 2009) and A/Turkey/OH/313053/2004 (H3N2) (Yassine et al., 2007) were propagated in Madin-Darby canine kidney (MDCK) cells. The H1N2 A/Swine/OH/FAH10-1/10 (H1N2-OH10) was used for CNPs-KAg vaccine preparation and H1N1 A/Swine/OH/24366/2007 (H1N1-OH7) was used for the virulent virus challenge infection. The H3N2 A/Turkey/OH/313053/2004 (H3N2-OH4) was used together with H1N2-OH10 and H1N1-OH7 for ex vivo cross-reactive immune analysis. The H1N2-OH10 vaccine virus and H1N1-OH7 challenge virus are heterologous to each other with 77% HA gene identity, whereas H3N2-OH4 virus, originally isolated from turkeys, is heterosubtypic to other two SwIAV with HA gene identity 63% (Yassine et al., 2007; Yassine et al., 2009; Ali et al., 2012). For vaccine preparation, cell culture fluid of H1N2-OH10 virus grown in MDCK cells was harvested and subjected to sucrose gradient ultracentrifugation. The virus pellet was suspended in phosphate-buffered saline (PBS), titrated for infectious virus titer and inactivated by using binary ethyleneimine (BEI) (Sigma, MO) as described previously (Dhakal et al., 2017b).

Preparation of Chitosan Based Nanovaccine and In Vitro Characterization

Chitosan NP loaded killed SwIAV antigen (KAg) (CNPs-KAg) formulation was prepared by the ionic gelation method as described previously (Calvo et al., 1997; Zhao et al., 2012; Mirzaei et al., 2017; Debnath et al., 2018) with some modifications. Briefly, 1.0% (w/v) low molecular weight chitosan polymeric (Sigma, MO) solution was prepared in an aqueous solution of 4.0% acetic acid under magnetic stirring until the solution became clear. The chitosan solution was sonicated; pH was adjusted to 4.3 and filtered via 0.44 μm syringe filter. Five mL of 1.0% chitosan solution was added to 5.0 mL deionized water and incubated with 3.0 mg SwIAV KAg dissolved in 1.0 mL 3-(N-morpholino) propanesulfonic acid (MOPS) buffer pH 7.4. Consequently, 2.5 mL of 1.0% (w/v) tripolyphosphate (TPP) (Sigma, MO) dissolved in 2.5 mL deionized water was added into the chitosan polymer solution with continuous magnetic stirring at room temperature (RT, 22° C.). The formulated SwIAV nanovaccine was centrifuged at 10,000 rpm for 10 min, dispersed in MOPS buffer pH 7.4, lyophilized with a cryoprotectant and stored at −80° C.

Particle size and zeta potential of empty and vaccine antigen loaded nanoparticles were measured after dispersion in physiological buffer saline (PBS, pH 7.4) and stored at 4° C. for at least 30 h by dynamic light scattering (DLS) method using a zeta-sizer coupled with an MPT-2 titrator (Malvern) as described previously (Lu et al., 2015). During each vaccination, CNPs-KAg nanoparticles were freshly prepared and used. The morphology of nanoparticles was obtained by using the cold field emission Hitachi S-4700 scanning electron microscope (SEM) (Dhakal et al., 2017b). Briefly, the powder form of nanoparticles was loaded on to aluminum stubs and coated with platinum prior to examination under the microscope. Protein loading efficiency in CNPs-KAg was estimated indirectly by determining the difference between initial amount of protein used for loading CNPs and the protein left in the supernatant (Zhao et al., 2012). In vitro protein release profile in CNPs-KAg suspended in PBS for up to 15 days was estimated and expressed as the cumulative percentage release of SwIAV antigen at each time point as described previously (Dhakal et al., 2017b). In brief, CNPs-KAg suspended in 500 μL PBS (pH 7.4) in triplicate in eppendorf tubes was incubated at 37° C. in a revolving roller apparatus. At indicated time point tubes were centrifuged, supernatant collected and pellet was resuspended in fresh 500 μL, PBS. Protein released in to the supernatant was estimated by micro BCA protein assay kit (Thermo Scientific, MA) and expressed as the percentage of cumulative protein released over the initial amount at time zero in particles.

In Vitro Uptake of CNPs-KAg by Antigen Presenting Cells (APCs)

Peripheral blood mononuclear cells (PBMCs) isolated from 9 to 10 week-old pigs were used for the in vitro antigen uptake study. Cells were suspended in enriched-Roswell Park Memorial Institute (E-RPMI) medium and seeded 1 million cells/well in 96 well cell culture plates. After overnight incubation at 37° C. in 5% CO₂, unattached cells were removed. The attached monocyte/macrophage cells were treated with SwIAV KAg or CNPs-KAg containing the antigen at 10 μg/mL concentration for 10 min, 30 min and 150 min. After the indicated period of incubation, the cells were fixed with 80% acetone, stained with IAV nucleoprotein-specific antibody (CalBioreagents, CA) followed by Alexa Fluor 488 conjugated goat anti-mouse IgG antibody (Life technologies, OR). Cells were evaluated under fluorescent microscope (Olympus IX70) and photomicrographs were taken (20×). For evaluation of SwIAV antigen uptake from CNPs-KAg treated porcine monocyte/macrophages prepared from three pigs PBMCs separately were incubated in 48-well plates seeded with 2×10⁶ cells per well overnight as described above. Cells were treated with KAg or CNPs-KAg at SwIAV antigen concentration 10 μg/mL for 10 min, 30 min and 150 min. Positive control was MDCK cells infected with SwIAV H1N2-OH10 at MOI 1 for 12 h. After indicated period of treatment cells were fixed using 1% paraformaldehyde (PFA), permeabilized and stained with IAV nucleoprotein-specific antibody (CalBioreagents, CA) followed by treatment with goat anti-mouse IgG Alexa Flour488 conjugated secondary antibody. Fifty thousand events were acquired in BD Aria II flow cytometer (BD Biosciences, CA) and analyzed the data by using the FlowJo software (Tree Star, OR).

In Vitro Generation and Stimulation of Porcine Dendritic Cells

Porcine monocyte derived dendritic cells (MoDCs) were prepared from PBMCs isolated from seven pigs as described previously (Nedumpun et al., 2016) with few modifications. Briefly, 25 million PBMCs per mL were seeded in each well of 6-well culture plates. After overnight incubation at 37° C. in a 5% CO₂ incubator, non-adherent cells were discarded and adhered cells were treated with GM-CSF (25 ng/mL) and IL-4 (10 ng/mL) cytokines. Half of the culture media was replaced on every third day. On day 7 the plate was centrifuged at 2000 RPM at 4° C. for 5 min and the supernatant was harvested gently, and generated immature MoDCs were stimulated in the same plate without seeding into fresh plates with medium only, LPS control (10 μg/mL), KAg (10 μg/mL) and CNPs-KAg containing 10 μg/mL of KAg for 48 h. The culture supernatant was harvested and estimated the levels of innate, proinflammatory and Th1 cytokines, IFN-α, TNF-α. IL-1β, IL-12, IL-6 and IL-10 by ELISA as described previously (Dwivedi et al., 2016).

Experimental Design

Caesarian-delivered colostrum-deprived (CDCD) and bovine colostrum-fed influenza antibody-free Large White-Duroc crossbred piglets were raised in a BSL2 animal facility at OARDC. Piglets at 4 weeks of age (male and female) were randomly assigned into one of the three experimental groups and kept in separate isolation rooms (Table 1).

TABLE 1
Experimental design showing different vaccine groups

| | | | | Day of |
|---|---|---|---|---|
| | | First | Second | challenge |
| Experimental | Pig | vaccination | vaccination | (DPV 35/ |
| groups | no. | (DPV 0) | (DPV 21) | DPC 0) |
| Unvaccinated | 3 | DMEM | DMEM | H1N1-OH7 |
| KAg | 4 | Inactivated | Inactivated | H1N1-OH7 |
| | | H1N2-OH10 | H1N2-OH10 | |
| CNPs-KAg | 5 | Inactivated | Inactivated | H1N1-OH7 |
| | | H1N2-OH10 | H1N2-OH10 | |
| | | encapsulated | encapsulated | |
| | | in chitosan | in chitosan | |
| | | nanoparticle | nanoparticle | |

The first IN vaccination was performed at 5 weeks of age and the second IN booster vaccination at 8 weeks of age. All piglets receiving virulent SwIAV were challenged at 10 weeks of age. Separate groups of pigs were vaccinated IN with DMEM (Gibco) or with 1×10⁷ TCID₅₀ equivalent of KAg or CNPs-KAg suspended in 2 mL DMEM by intranasal mist as described previously (Dhakal et al., 2017b). The challenge infection was done using heterologous H1N1-OH7 SwIAV (6×10⁶ TCID₅₀) in 2 mL, divided into 1 mL administered IN and 1 mL intratracheally as described previously (Hiremath et al., 2016; Dhakal et al., 2017a; Dhakal et al., 2017b).

Serum samples were collected at days post-vaccination (DPV) 21 and 35. The rectal temperatures were recorded daily from day post-challenge (DPC) 0 onward and nasal swab samples were collected at DPC 0, DPC 4 and DPC 6. Pigs were euthanized at DPC 6 and serum and bronchoalveolar lavage (BAL) fluid were collected. During necropsy, lungs were examined for macroscopic pneumonic lesions and scored as described previously (Dhakal et al., 2017b). Lung lysates were prepared by homogenization of 1.0 g of lung tissue collectedly from the right apical lobe (Dhakal et al., 2017b). Nasal swabs, sera, BAL fluid and lung lysate samples were stored at −80° C. until processed for antibody and virus titration. The PBMCs were isolated from blood at DPV 35/DPC 0 and DPC 6 (Dhakal et al., 2017b). Mononuclear cells were harvested from tracheobronchial lymph nodes (TBLN-MNCs) at DPC 6 as described previously (Dwivedi et al., 2016).

Antibody Titration

Hemagglutination inhibition (HI) antibody titers against IAVs H1N1-OH7, H1N2-OH10 and H3N2-OH4 in sera and BAL fluid samples were determined as described previously (Dhakal et al., 2017b). The SwIAV-specific IgG and IgA antibodies in nasal swabs, sera, BAL fluids and lung lysates were determined by ELISA (Dhakal et al., 2017b). Briefly, 96 well plates (Greiner bio-one, NC) were coated overnight with respective pre-titrated IAV antigen (5 μg/mL) and blocked with 5% skim milk powder containing 0.05% Tween-20 for 2 hr at RT. After wash five-fold dilutions of nasal swab, serum, BAL fluid and lung lysate samples in in PBS containing 2.5% skim milk powder and 0.05% Tween-20 were added to marked duplicate wells, incubated for 2 h at RT, washed and horse radish peroxidase (HRP)-conjugated goat anti-pig IgA (Bethyl Laboratories Inc., TX) or goat anti-pig IgG (KPL, MD) was added. Finally, the antigen and antibody interaction was detected by using 1:1 mixture of peroxidase substrate solution B and TMB peroxidase substrate (KPL, MD). The reaction was stopped using 1.0 M phosphoric acid and optical density (OD) was measured at 450 nm using a Spectramax microplate reader (Molecular devices, CA).

Antigen Specific Cell Proliferation Assay

The PBMCs isolated at DPV 35/DPC 0 were cultured together with H1N1-OH7, H1N2-OH10 or H3N2-OH4 SwIAV at 0.1 multiplicity of infection (MOI) and incubated at 37° C. in 5% CO₂ incubator for 72 h. Antigen-specific lymphocyte proliferation was determined by using the cell titer 96 aqueous non-radioactive proliferation assay kit (Promega, WI). The cell proliferative response was compared among groups using lymphocyte stimulation index values as described previously (Dhakal et al., 2017b).

Cytokine ELISA

PBMCs isolated at DPC 0 and TBLN-MNCs at DPC 6 were cultured with H1N2-OH10, H1N1-OH7 or H3N2-OH4 SwIAV at 0.1 MOI. After 72 h of stimulation the supernatant was collected and interferon gamma (IFNγ) secretion was determined by ELISA as described previously (Dhakal et al., 2017b). Similarly, production of interleukin-6 (IL-6) in BAL fluid collected at DPC 6 was determined by ELISA (Dwivedi et al., 2016).

Virus Titration

Viral titers contained in nasal swabs and BAL fluids were determined in ten-fold dilution of the samples in DMEM containing TPCK-trypsin (1 μg/mL). The samples were transferred to quadruplicate 96 well cell culture plates wells containing overnight cultured monolayers of MDCK cells and incubated for 72 h, 37° C., 5.0% CO₂. Cells were fixed with acetone and immunostained with IAV nucleoprotein-specific antibody (# M058, CalBioreagents, CA) followed by Alexa Fluor 488 conjugated goat anti-mouse IgG (H+L) antibody (Life technologies, OR). Virus replication in cells was determined by using immunofluorescence technique as described previously (Dhakal et al., 2017b).

Histopathology of Lungs

For histopathological analysis of pulmonary tissues, 10% formalin-inflated apical, cardiac and diaphragmatic lobes were collected and further emulsion-fixed in 10% neutral buffered formalin. Five μm sections of formalin-fixed, paraffin embedded apical, cardiac and diaphragmatic lung lobes were stained with hematoxylin and eosin (HE) as previously described (Dhakal et al., 2017b). The H&E-stained tissues sections were examined for microscopic changes of interstitial pneumonia, peribronchial and perivascular accumulation of mononuclear cells, bronchial exudates and epithelial changes related to influenza infection. All these parameters were scored by a board certified veterinary pathologist (SK) who was not provided with any vaccination history of pig groups in a scale of 0 (no change compared from normal) to 3 (marked changes from normal) as described previously (Dhakal et al., 2017b).

Flow Cytometry

The PBMCs isolated at DPC 0 and TBLN-MNCs at DPC 6 were immunostained for T lymphocyte subset phenotyping as described previously (Dhakal et al., 2017b). Antibodies used in the flow cytometry were: anti-porcine CD3 (Southernbiotech, AL), CD4a (Southernbiotech, AL), CD8a (Southernbiotech, AL) and CD8β (BD Biosciences, CA). Briefly, the cells were blocked with 2% pig serum in fluorescence activated cell sorting (FACS) buffer and surface labelled with pig lymphocyte-specific purified, biotin or fluorochrome-conjugated antibodies or their respective antibody isotypes. Cells were fixed using 1% paraformaldehyde, washed, suspended in FACS buffer and acquired using BD Aria II flow cytometer (BD Biosciences, CA). Data analysis was done using FlowJo software (Tree Star, OR).

Quantitative Reverse Transcription PCR (RT-qPCR)

Total RNA was extracted from PBMCs at DPC 0 and TBLN at DPC 6 using TRIzol reagent (Invitrogen, CA) as per the manufacturer's instructions. NanoDrop™ 2000c Spectrophotometer (Thermo Fisher Scientific, MA) was used to determine the concentration and purity of RNA. cDNA was prepared from 1 μg of total RNA using the QuantiTect Reverse Transcription Kit (AIAGEN). Primers of housekeeping gene (β actin) and target genes (T-bet and GATA-3) used in this experiment were described previously (Meurens et al., 2009). The mRNA expression was analyzed by 7500 Real-Time qPCR system (Applied Biosystems, CA) using the qScript™ One-Step SYBR Green qRT-PCR kit, Low ROX™ (Quantabio, MA). The target gene expression level was normalized with housekeeping gene levels and the fold change was determined by comparative 2^{-ΔΔC}T method (Livak and Schmittgen, 2001).

Statistical Analysis

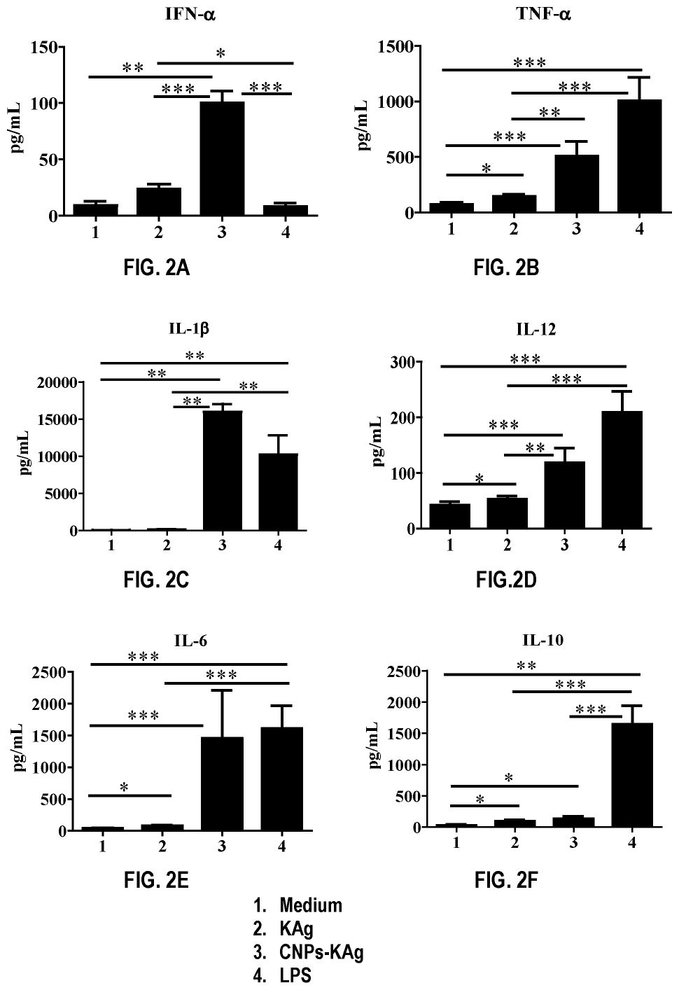

Statistical analysis was performed by using non-parametric Kruskal-Wallis test followed by Dunn's post-hoc test using the software GraphPad Prism 5 (GraphPad Software, Inc., CA). Pig rectal temperature data was analyzed by repeated measure ANOVA using Friedman test followed by Dunn's pairwise comparison. Cytokine data (FIG. 2) between two groups were analyzed by Mann-Whitney test. A p-value of less than 0.05 was considered statistically significant. The infectious virus titer was determined using Reed and Muench method. Data were presented as the mean±SEM of 3-5 pigs except for the HI titers which were expressed as geometric mean with 95% confidence interval.

### Results

In this study, chitosan was used as a mucoadhesive polymer for encapsulation of influenza KAg (CNP-KAg), and heterologous vaccine-challenge trials were performed in nursery pigs. Due to its cationic nature, chitosan binds readily to the mucosal surface and possesses adjuvant properties which helps in immune activation. In this example, it is shown that CNP-KAg significantly improved mucosal IgA responses, resulting in reduced nasal virus shedding and improved cell-mediated immunity against SwIAV of different subtypes.

Characterization of CNPs-KAg Vaccine

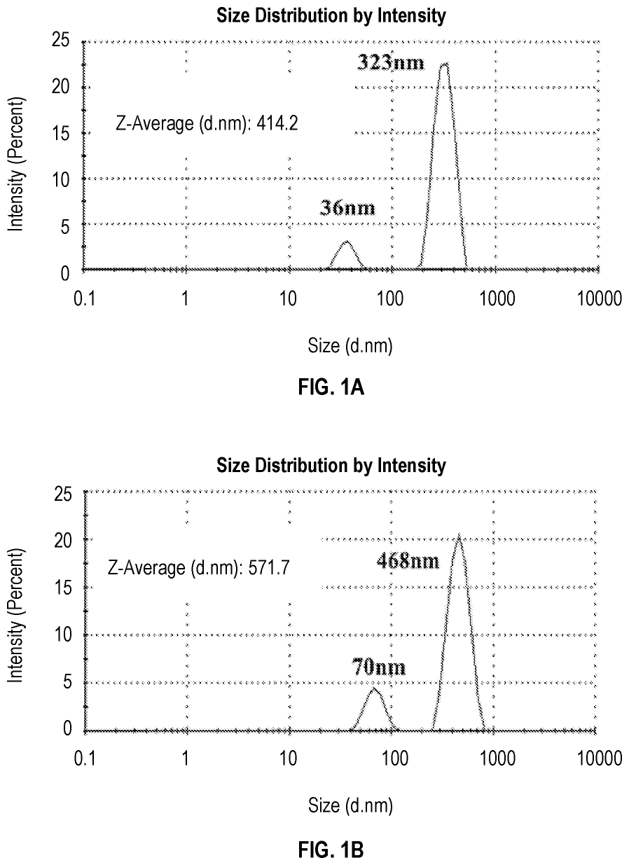

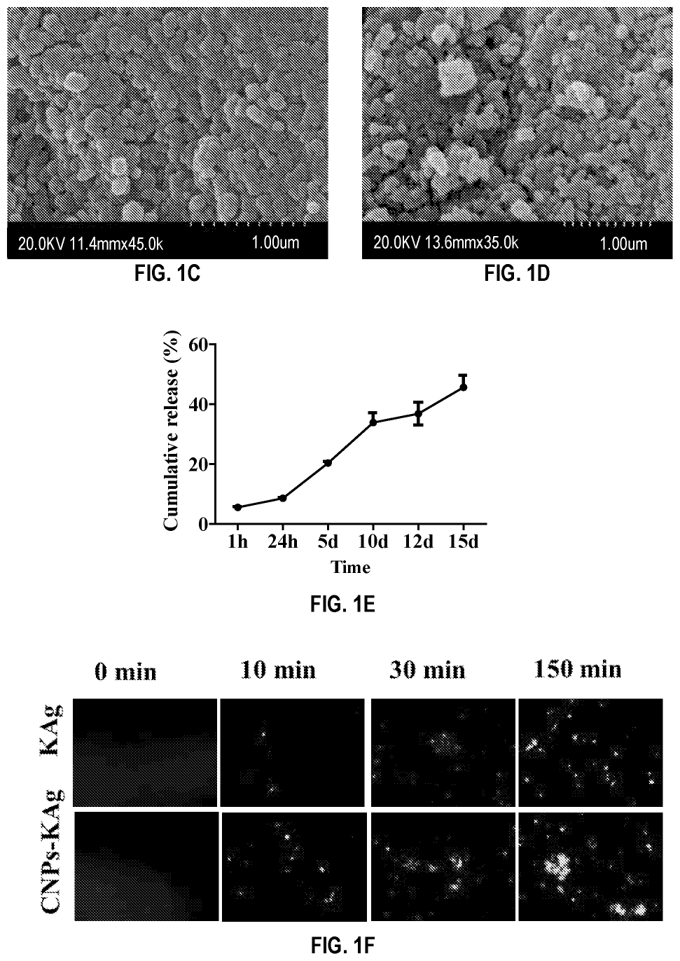

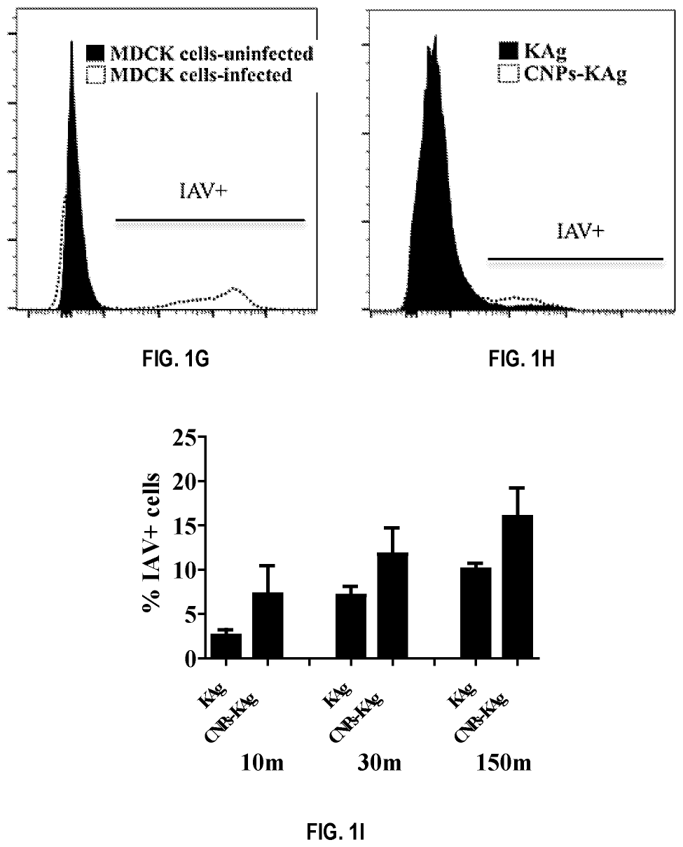

The encapsulation efficiency of SwIAV KAg in chitosan nanovaccine formulation was 67%. This result was comparable to encapsulation efficiency of chitosan NPs entrapped with Salmonella outer membrane protein antigens (70%) (Renu et al., 2018, manuscript under review). As determined by DLS, the average size of the empty (FIG. 1A) and antigen loaded (FIG. 1B) NPs was 414.2 nm and 571.7 nm, respectively. Empty NPs showed two peaks at 36 nm (˜10%) and 323 nm (˜90%) with polydispersity index (PDI) 0.39. Likewise, antigen loaded NPs also had two peaks at 70 nm (˜15%) and 468 nm (˜85%) with PDI 0.60. Data shows that the CNPs-KAg nanoparticles were polydispersed in nature. SEM analysis showed the morphology of the empty NPs were spherical with smooth surface (FIG. 1C), while antigen loaded NPs had relatively rough and irregular surface (FIG. 1D). The surface charge of empty and antigen loaded chitosan NPs was +1.88 mV and +1.69 mV, respectively. A 6% burst release was observed, i.e., surface associated antigen release during the first 1 h, and on an average 9% of antigen was released after 24 h of incubation. Further, slow and sustained release of antigen was observed with cumulative release of approximately 46% after 15 days (FIG. 1E).

To determine whether chitosan encapsulation of KAg enhances the uptake of antigen by APCs, monocyte/macrophages were prepared from PBMCs and allowed for interaction with KAg or CNPs-KAg and stopped the reaction at three different time points. Internalization of CNPs-KAg vaccine by monocytes/macrophages was observed within 10 min of treatment indicated by higher number of influenza-specific fluorescent signals compared to KAg treatment (FIG. 1F). Further the uptake of CNPs-KAg was substantially increased after 30 min and 150 min post-treatment compared to control KAg-treated cells. Flow cytometry analysis of monocyte/macrophages treated with KAg or CNPs-KAg was performed to determine the frequency of specific uptake of influenza antigens in APCs. In soluble KAg treated cells an average 2.7%, 7.1% and 10.1% cells positive for influenza antigen, and in CNPs-KAg treated cells 7.2%, 11.7% and 16% cells with uptaken influenza antigen after 10 min, 30 min and 150 min of incubation were noticed, respectively (FIG. 1I). This data clearly demonstrated that CNPs-KAg was efficiently internalized by pig APCs better than soluble antigens.

CNPs-KAg Formulation Induced the Secretion of Cytokines by Porcine MoDCs In Vitro

In order to elucidate the adjuvant property of chitosan NPs in porcine APCs, porcine MoDCs were treated with medium and LPS as control to compare the effect of soluble KAg and CNPs-KAg treatment in inducing secretion of different cytokines. The medium control cells had very little secretion of all the detected cytokines, while LPS treatment induced the production of all the analyzed cytokines except IFN-α (FIG. 2A-2F). Cells treated with KAg secreted significantly higher levels of proinflammatory cytokines TNF-α (FIG. 2B) and IL-6 (FIG. 2E) and Th1 cytokine IL-12 (FIG. 2D) compared to medium control. In DCs treated with CNPs-KAg production of innate IFN-α (FIG. 2A), TNF-α (FIG. 2B), IL-1β (FIG. 2C) and IL-12 (FIG. 2D) were significantly higher in CNPs-KAg treated compared to soluble KAg treated cells. In CNPs-KAg treated cells production of IL-6 (FIG. 2E) and Th2 cytokine IL-10 (FIG. 2F) were higher than medium control cells, but not significantly higher compared to KAg treated cells. In vitro DCs treatment data shows that chitosan nanovaccine formulation has potent adjuvant effect on porcine DCs.

CNPs-KAg Vaccine Augmented the IAV Specific Mucosal Antibody Response in the Respiratory Tract of Pigs

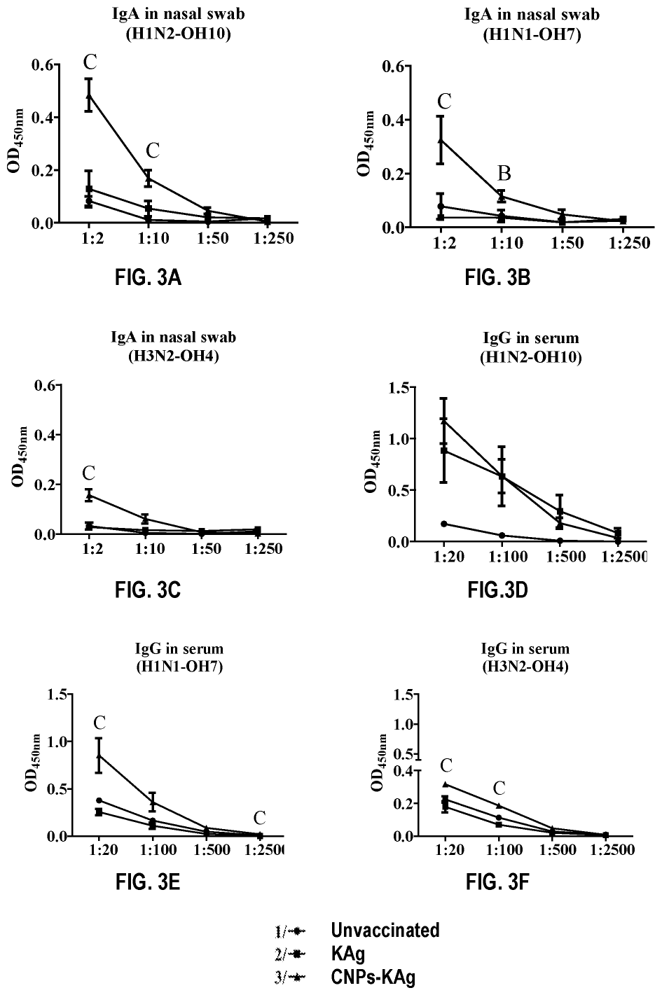

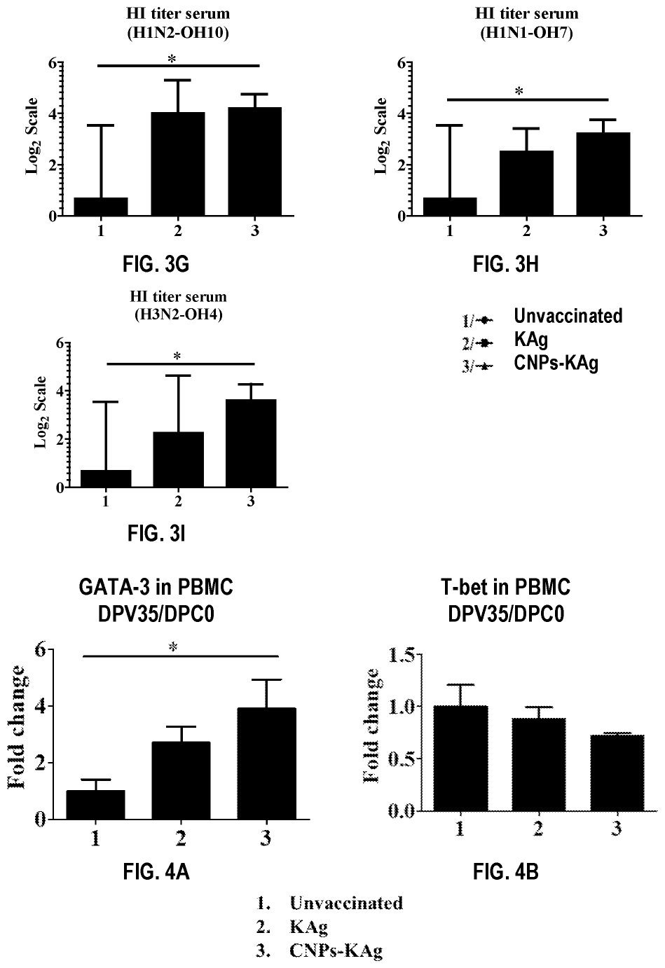

Secretary IgA antibody levels in nasal swab samples collected after IN prime-boost vaccination at DPV 35/DPC 0 was significantly higher (p<0.05) in CNPs-KAg-vaccinated pigs compared to pigs receiving soluble KAg when tested against the homologous H1N2-OH10 (FIG. 3A), heterologous H1N1-OH7 (FIG. 3B) and heterosubtypic H3N2-OH4 IAVs (FIG. 3C). A significant difference in antibody response was observed between CNPs-KAg and KAg-vaccinates in serial two-fold diluted nasal swab samples (FIG. 3A-3C). This data suggests that the CNPs-KAg IN delivery induced enhanced cross-reactive mucosal secretary IgA antibody response in pigs. Specific IgG antibody response in sera after prime-boost vaccination in KAg vaccinated pigs against the vaccine virus was comparable to the CNPs-KAg vaccine group (FIG. 3D). While significantly higher (p<0.05) cross-reactive IgG response was observed in CNPs-KAg vaccinates against heterologous H1N1-OH7 (FIG. 3E) and heterosubtypic H3N2-OH4 (FIG. 3F) IAVs compared to KAg vaccinated animals. In CNPs-KAg vaccinated pig sera, IAV-specific HI antibody titers against H1N2-OH10 (FIG. 3G), H1N1-OH7 (FIG. 3H) and H3N2-OH4 (FIG. 3I) were significantly higher (p<0.05) compared to mock pig group. The HI titers in CNPs-KAg-vaccinates were around 2-fold higher compared to KAg-vaccinates against heterologous (FIG. 3H) and heterosubtypic (FIG. 3I) IAVs, but the data was not statistically significant (p>0.05). The expression of T helper 2 (Th2) specific transcription factor GATA-3 mRNA in PBMCs of pigs at DPV 35/DPC 0 was 4-fold and 1.5-fold higher in CNPs-KAg vaccinated pigs compared to unvaccinated control (p<0.05) and KAg-vaccinated pigs (p>0.05) (FIG. 4A). The expression of Th1 specific transcription factor T-bet in PBMCs was not significantly different among the pig groups (FIG. 4B).

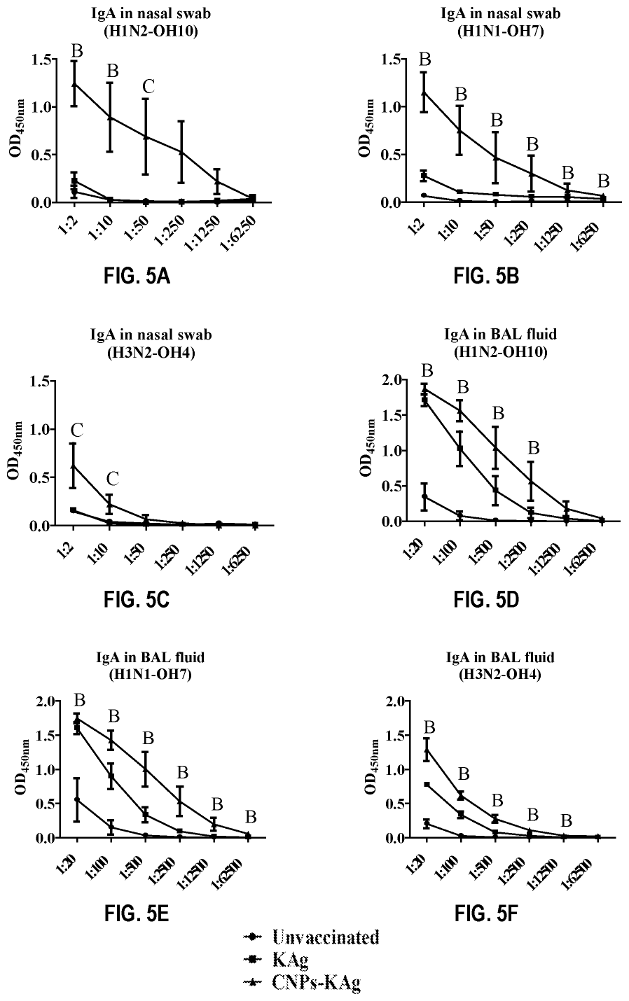

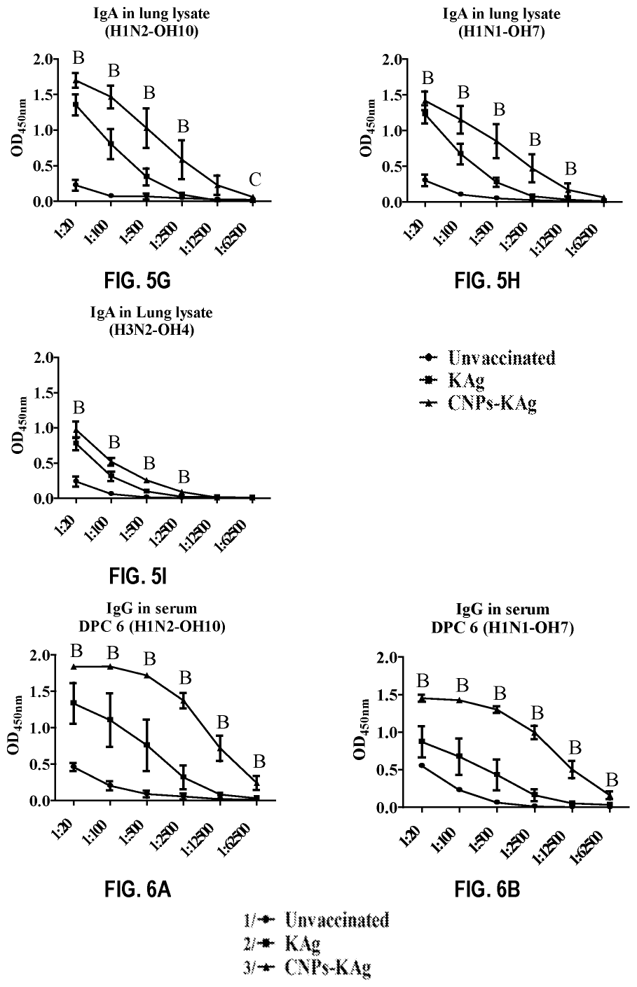

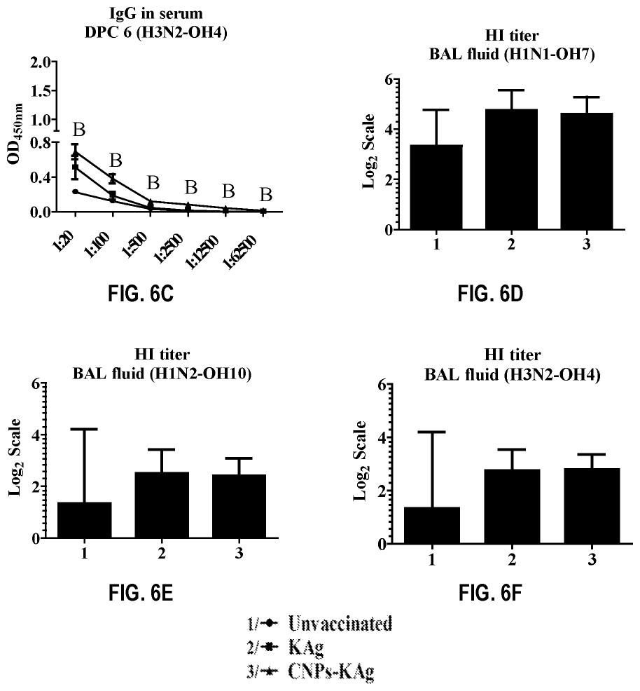

The mucosal IgA response in pigs post-challenge at DPC 6 was determined and the data indicate that specific IgA in CNPs-KAg vaccinated pig group was significantly higher (p<0.05) compared to unvaccinated challenged animals and remarkably higher compared to KAg vaccinated and challenged animals against all three IAV subtypes in nasal swabs (FIG. 5A, B, C), BAL fluids (FIG. 5D, E, F) and lung lysates (FIG. 5G, H, I). These data indicated the secretion of robust mucosal IgA antibody in the upper respiratory tract (nasal swabs), lower respiratory tract (BAL fluids) and lung parenchyma (lung lysates) of pigs. Similarly, systemic IgG antibody response in serum at DPC 6 was also enhanced in the CNPs-KAg-vaccinates compared to unvaccinated (p<0.05) and KAg vaccinated and virus challenged animals (FIG. 6A, B, C). However, HI antibody titers in BAL fluid at DPC 6 were comparable between KAg and CNPs-KAg vaccinates (FIG. 6D, E, F).

CNPs-KAg Vaccine Enhanced Systemic Specific Cell-Mediated Immune Response Against IAVs

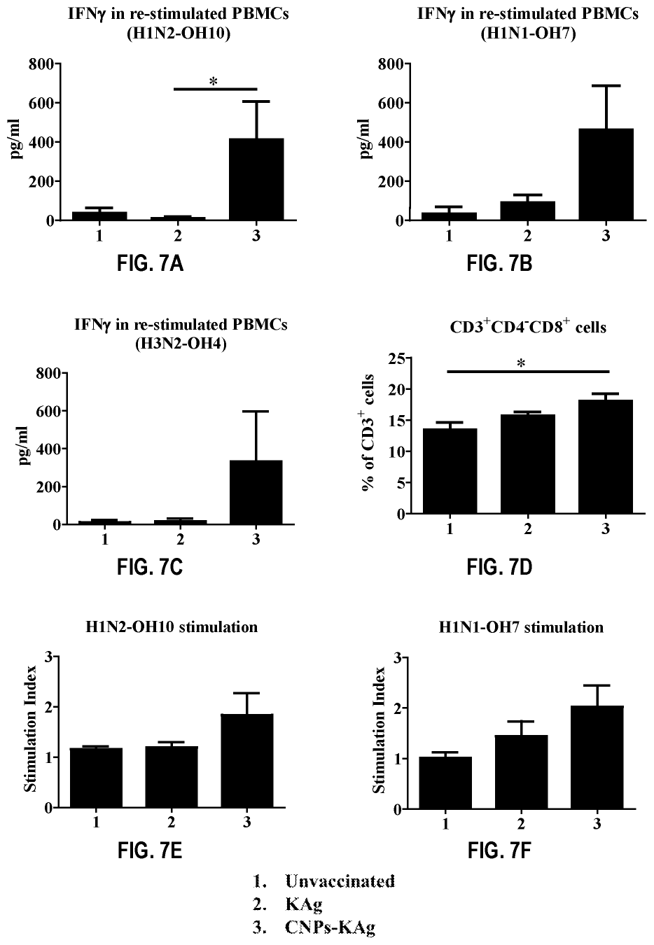

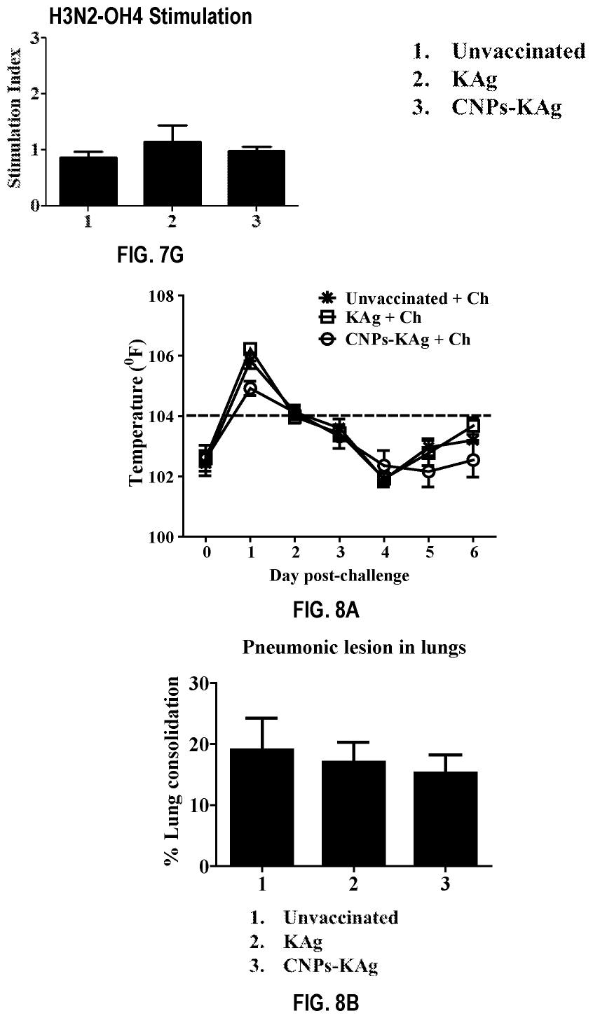

To understand the role of chitosan delivered IAV nanovaccine in induction of specific cell-mediated immune response after IN vaccinations, isolated PBMCs at DPV 35 were restimulated with H1N2-OH10, H1N1-OH7 and H3N2-OH4 viruses. The harvested cell culture supernatants were analyzed for IFNγ secretion and significantly higher (p<0.05) levels of IFNγ in homologous H1N2-OH10 virus restimulated CNPs-KAg compared to soluble KAg vaccinated pigs was observed (FIG. 7A). Though not statistically significant, the IFNγ recall response in heterologous and heterosubtypic viruses restimulated cells were noticeably higher levels in CNPs-KAg vaccinates than in KAg-vaccinated pigs (FIG. 7B, C). The average IFNγ amounts in CNPs-KAg vaccine group against H1N1-OH7 and H3N2-OH4 viruses was 463 pg/mL and 332 pg/mL compared to 91 pg/mL and 16 pg/mL in KAg vaccinates, respectively (FIG. 7B, C). Phenotyping of PBMCs isolated at DPC 0 was performed (by flow cytometry). The frequency of cytotoxic T cells (CTLs) in CNPs-KAg vaccinated pigs (average: 18.1%) was higher compared to KAg-vaccinated (average: 15.7%, p>0.05) and unvaccinated pigs (average: 13.4%, p<0.05) (FIG. 7D). This finding is consistent with enhanced IFNγ response in CNPs-KAg vaccinated pigs, as activated CTLs are one of the major T cell subsets which secrete high levels of antiviral cytokine IFNγ. In addition, in PBMCs at DPC 0 virus specific cell proliferation was detected in an increased trend upon restimulation with homologous (FIG. 7E) and heterologous (FIG. 7F), but not with heterosubtypic (FIG. 7G) viruses in CNPs-KAg vaccinated pigs. Overall, these data suggested the presence of superior cross-reactive effector memory lymphocyte response in pigs induced by chitosan encapsulation of inactivated SwIAV antigen.

CNPs-KAg Vaccine Reduced the Inflammatory Changes in the Lungs of Virulent and Heterologous Virus Challenged Pigs

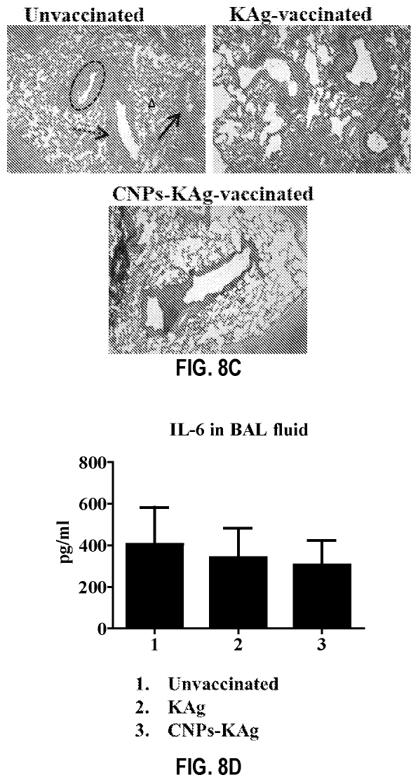

Rectal temperature of pigs was recorded daily post-challenge until euthanized. Pigs in all groups had fever (≥104° F.) for first two days after challenge. However, there was no statistical difference in temperature profile among the pig groups (FIG. 8A). Macroscopic pulmonary lesions were scored for percent consolidation induced by influenza infection and observed lower pulmonary consolidation in CNPs-KAg vaccinates (mean score 15) compared to KAg (mean score 17) and unvaccinated animals (mean score 19) (FIG. 8B), but the data was not statistically significant (p>0.05). Microscopic pulmonary lesions were subjectively scored on Hematoxylin and Eosin (H&E) stained lung sections where a score of 0=no change from normal, 1=minimal change from normal, 2=moderate change from normal and 3=severe change from normal (FIG. 8C). The mean scores of interstitial pneumonia (2, 1.6 and 0.8), peribronchial inflammation (2, 1.8 and 1.8), perivascular inflammation (1.6, 0.3 and 0.5), bronchial exudates (0.7, 0.1 and 0.2) and epithelial changes (0.3, 0.3 and 0.1) were observed in virus challenged unvaccinated, KAg and CNPs-KAg-vaccinates, respectively. All the microscopic evaluation of pulmonary tissues was conducted by a board certified veterinary pathologist. A moderate reduction in inflammatory changes was observed in both the vaccinated pig groups when compared to the lesion scores in the unvaccinated and challenged group. In particular, the interstitial pneumonia and epithelial changes were much reduced in CNPs-KAg group compared to soluble KAg vaccinated pigs.

The levels of proinflammatory cytokine IL-6 secretion was evaluated in the BAL fluid and relatively lower levels were observed in CNPs-KAg vaccinated pigs, consistent with the lower macroscopic and microscopic lung lesions (FIG. 8D).

CNPs-KAg Vaccine Enhanced the Mucosal Cellular Immune Response in the Tracheobronchial Lymph Nodes of Virulent and Heterologous Virus-Challenged Pigs

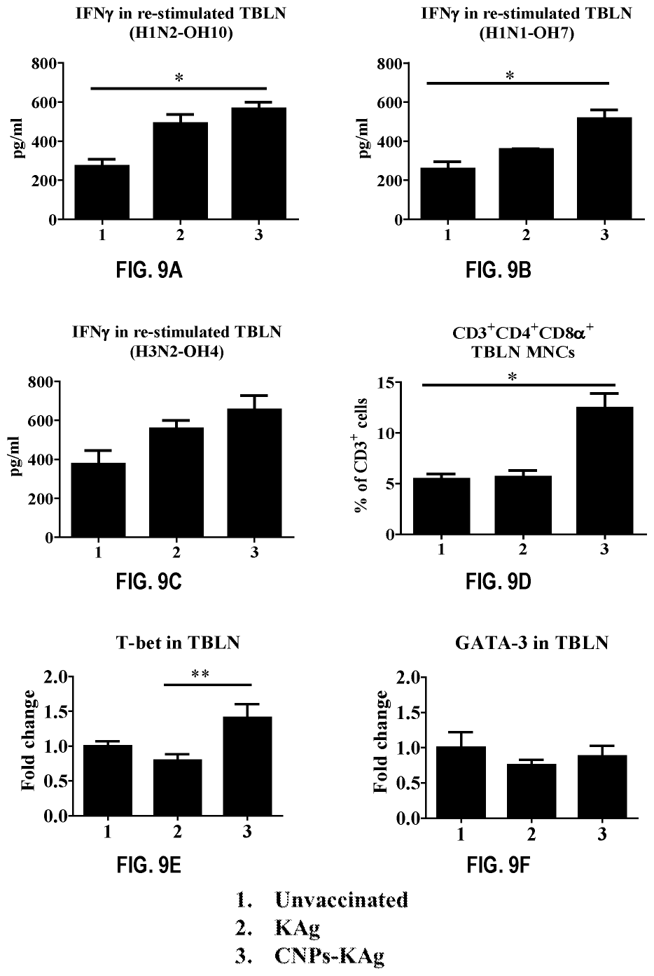

In the CNPs-KAg vaccinated pigs, the frequency of CTLs, IFNγ and specific lymphocyte proliferation index values were augmented in PBMCs (FIG. 7). The cell-mediated immune response in the lung draining TBLN was also examined in these vaccinated pigs. These data demonstrated significantly higher (p<0.05) secretion of IFNγ by TBLN-MNCs restimulated with vaccine (H1N2-OH10) and challenge (H1N1-OH7) viruses in CNPs-KAg, but not in KAg vaccinated compared to mock group (FIG. 9A,B). Cells similarly stimulated with heterosubtypic (H3N2-OH4) IAV, showed an increase in IFNγ secretion in CNPs-KAg vaccinated pig group but this increase was not statistically significant (FIG. 9C). Flow cytometry analysis of TBLN-MNCs isolated at DPC 6 was performed and a significantly higher (p<0.05) frequency of T helper/memory cells (CD3⁺CD4⁺CD8α⁺) was observed, one of the principle contributor of IFNγ production in pigs (Hiremath et al., 2016), in CNPs-KAg vaccinated pig group compared to unvaccinated and challenged animals (FIG. 9D). The expression of Th1 and Th2 transcription factors mRNA level in TBLN collected at DPC 6 were analyzed. Consistent with augmented cellular response in TBLN-MNCs of CNPs-KAg vaccinated pigs, in frozen TBLN tissues mRNA expression of the Th1 specific transcription factor T-bet was significantly higher (p<0.05) in CNPs-KAg compared to KAg vaccinates (FIG. 9E). The expression of Th2 transcription factor GATA-3 mRNA was not increased in TBLN.

CNPs-KAg Vaccine Reduced Virus Shedding in the Nasal Cavity and Also Pulmonary Viral Titers in SwIAV Challenged Pigs

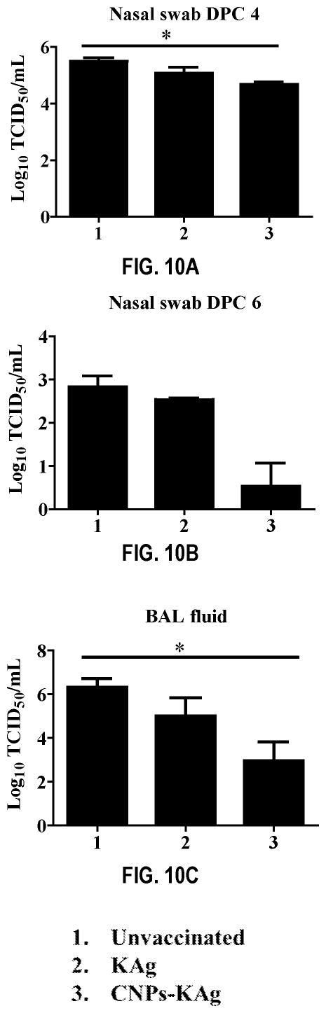

Significantly reduced (p<0.05) challenge virus shedding was observed at DPC 4 from the nasal passage of CNPs-KAg vaccinates compared to unvaccinated and challenged animals (FIG. 10A). By DPC 6, infectious virus was detected in the nasal passage of only 1 of 5 pigs (20%) vaccinated with CNPs-KAg vaccine, while all pigs in KAg-vaccinated and unvaccinated groups were shedding virus ranging from 10^{2.5} to 10^{3.3} TCID₅₀/mL (FIG. 10B). The average virus titers in nasal swab at DPC 6 in unvaccinated, KAg- and CNPs-KAg vaccinated and challenged pigs were 10^{2.8}, 10^{2.5} and 10^{0.5} TCID₅₀/mL, respectively (FIG. 10B). Similarly, virus titer in BAL fluid on DPC 6 was significantly reduced (p<0.05) in CNPs-KAg but not in KAg group compared to unvaccinated virus challenge pigs (FIG. 10C). The average virus titers in BAL fluid at DPC 6 in unvaccinated, KAg- and CNPs-KAg vaccinated and IAV challenged pig groups were 10^{6.3}, 10⁵ and 10³ TCID₅₀/mL, respectively.

### Discussion

Chitosan is a natural polymer synthesized by deacetylation of chitin, one of the most abundant polysaccharides in nature (Illum et al., 2001). Chitosan forms an attractive excipient for drug and vaccine delivery as it bears biocompatible, biodegradable, mucoadhesive, polycationic and immunomodulatory properties (Illum et al., 2001; Wang et al., 2011). Chitosan is often coupled with tripolyphosphate (TPP), a polyanion that helps in encapsulation of the biochemical agents through inotropic gelation. The chitosan and TPP (CS/TPP) NPs formulation in mice was shown to induce both cell-mediated (Th1) and humoral (Th2) immune responses when immunized through IN route against Streptococcus equi (Figueiredo et al., 2012). Similarly, tetanus toxoid loaded in CS/TPP NPs IN delivered in rat were efficiently transported through the nasal epithelium, and in mice it induced long-lasting systemic and mucosal antibody response compared to soluble antigen (Vila et al., 2004). Mice immunized through IN route using CS/TPP based influenza split virus vaccine was shown to induce higher systemic and mucosal antibody response than soluble antigens, and also enhanced the cell-mediated immune response indicated by increased IFNγ secreting cells frequency in spleen (Sawaengsak et al., 2014). Unlike the preparation of poly(lactic-co-glycolic) acid (PLGA) and polyanhydride nanoparticles, the process of preparing chitosan nanoparticles does not need any organic solvents and thus involves a simple and mild procedure protecting sensitive biochemical agents including proteins and provides scope for easy modification of particles (Astete and Sabliov, 2006; Torres et al., 2006; Peniche and Peniche, 2011; Rampino et al., 2013).

In this example, chitosan-based influenza nanovaccine was prepared using TPP by ionotropic gelation technique. The resulting NPs were around 500 nm in diameter which is adequate for efficient uptake by APCs (Foged et al., 2005; Hiremath et al., 2016; Dhakal et al., 2017a; Dhakal et al., 2017b). The size of NPs was slightly increased after antigen loading like reported earlier (Satzer et al., 2016). But the surface charge of the NPs did not change much with or without antigen loading, and the charge (+2.84 mV) was comparable to NPs entrapped with Newcastle disease virus (NDV), which was also loaded in CS:TPP at 2:1 ratio formulation like the CNPs-KAg (Zhao et al., 2012). The stability of CNPs-KAg nanoparticles suspended in physiological buffer until 30 h (kept at 4° C.) was evaluated. For vaccination of pigs, CNPs-KAg was freshly prepared and maintained on ice until delivered IN (1-2 h) which ensured stability of NPs vaccine. For better stability and long-term storage of nanoparticle vaccines, the surface charge should be highly negative or positive (Illum et al., 2001; Yu and Xie, 2012). But the CNPs-KAg nanoparticles were polydispersed in nature and had weak positive surface charge, suggesting that further improvements to the CNPs-KAg formulation is required through optimization of the ratio of CS:TPP:KAg to ensure better physicochemical properties. The optimal CS:TPP:KAg combination should yield higher positive surface charge, monodispersed nature, relatively smaller size NPs (100 to 300 nm) and stable for long time at different storage conditions. The encapsulation efficiency of KAg in chitosan NPs formulation was 67%, higher than the encapsulation efficiency of H1N2-OH10 KAg (˜50-55%) in PLGA and polyanhydride NPs (Dhakal et al., 2017a; Dhakal et al., 2017b). The higher encapsulation efficiency of vaccine antigens is desirable to reduce the cost of vaccine production. The protein release from CNPs-KAg was slower than previously reported similar CS/TPP NPs formulation, wherein close to 50% of NDV antigens were released from CNPs within first three days (Zhao et al., 2012). Chitosan nanoparticle encapsulation enhances the antigen uptake by APCs, increases expression of activation markers and secretion of proinflammatory cytokines by APCs (Koppolu and Zaharoff, 2013). SwIAV antigens delivered in chitosan NPs were efficiently internalized by porcine APCs compared to soluble KAg; and importantly, induced the production of innate, proinflammatory and Th1 cytokines compared to soluble KAg.

Induction of strong mucosal immunity is associated with increased breadth of protective efficacy against influenza, and inactivated IM vaccines do not elicit high levels of antigen-specific mucosal IgA antibody response in the respiratory tract (Tamura and Kurata, 2004; van Riet et al., 2012). Moreover, IM influenza vaccines in pigs have a limitation of not being effective in presence of maternal derived antibody (MDA) (Wesley and Lager, 2006). However, successful IN vaccination has a potential to overcome MDA interference because of induction of robust local mucosal immunity in the respiratory tract with minimal MDA interference (Zhang et al., 2016). Chitosan is an attractive polymer for intranasal immunization (van der Lubben et al., 2001). It enhances the absorption of vaccine particles across the nasal epithelium (Illum et al., 1994). Further, when compared to aqueous chitosan solution, insulin-loaded chitosan nanoparticles (300-400 nm diameter) increased the nasal absorption of insulin (Fernandez-Urrusuno et al., 1999). Due to the positive charge of chitosan, it can interact with anionic components such as sialic acid of glycoproteins on epithelial cell surfaces thereby prolonging—local retention time and decreasing antigen clearance on mucosal surfaces. In addition to its bioadhesive properties, chitosan enhances paracellular and intracellular transport of particulate antigens into the subepidermal space for optimal contact with APCs and other cells associated with immune responses (Artursson et al., 1994; Dodane et al., 1999). In mice, intranasal delivery of chitosan nanoparticle-based hepatitis B vaccine enhances the mucosal IgA antibody response (Borges et al., 2008; Lebre et al., 2016). Other murine studies have shown that intranasal immunization with chitosan-based nanovaccine formulations induce robust mucosal and systemic antibody responses against Pneumococcus spp., Diphtheria spp. and Bordetella spp. (Jabbal-Gill et al., 1998; McNeela et al., 2000; Xu et al., 2011).

An influenza subunit vaccine coadministered intranasally with chitosan delivery system enhanced both mucosal and systemic antibody response in mice (Bacon et al., 2000). Intranasal delivery of chitosan delivered DNA vaccine against Coxsackievirus in mice enhanced the secretion of both serum IgG and mucosal IgA as wells as CTLs activity in spleen (Xu et al., 2004). Consistent with the previous studies in mice (Renegar and Small, 1991; Jabbal-Gill et al., 1998; McNeela et al., 2000; Xu et al., 2011), the prime-boost vaccination of CNPs-KAg in pigs improved the IgA antibody secretion in the nasal passage and lungs. Importantly, robust secreted antibodies were cross-reactive against heterologous and heterosubtypic IAV and helped in significant reduction in nasal virus shedding and lung load of a heterologous challenge virus. In a previous experiment, PLGA-SwIAV KAg nanovaccine failed to reduce nasal virus shedding in spite of inducing robust specific cell-mediated immune response and reducing virus load in the lungs of most of the pigs. This anomaly was likely due to the inability of PLGA-encapsulated vaccine to induce mucosal IgA response (Dhakal et al., 2017b). Similarly, polyanhydride-SwIAV KAg nanovaccine also enhanced specific cell-mediated immunity but did not enhance mucosal antibody responses and hence did not significantly reduce the nasal virus shedding (Dhakal et al., 2017a). Like earlier murine studies (Etchart et al., 1996; Bergquist et al., 1997), intranasal vaccination with CNPs-KAg also induced influenza-specific systemic IgG antibody and HI titers.

Cell-mediated immunity is of prime importance for providing complete protection against intracellular pathogens. The Th1 cytokine IFN-γ is a critical cytokine involved in antiviral responses (Samuel, 2001; La Gruta and Turner, 2014). Chitosan is superior to alum adjuvant in enhancing the cell-mediated immune responses (Mori et al., 2012). It also induces type I IFN secretion from immature dendritic cells (DC) which helps in DC maturation and generation of Th1 mediated cellular immune responses (Carroll et al., 2016). In this study, enhanced IFNγ secretion by activated lymphocytes in a recall response with genetically variant IAVs was observed in both PBMCs and TBLN-MNCs of CNPs-KAg-vaccinated pigs. The observed spike in IFNγ recall response was associated with enhanced virus-specific cellular response both at mucosal sites and systemically. Activated T cell subsets such as T helper and CTLs and innate NK cells are the sources of IFNγ (Farrar and Schreiber, 1993). The prime-boost vaccination schedule employed in this study with CNPs-KAg increased the CTLs in PBMC cultures, the major source of IFNγ, the cytokine that clears virus from infected cells (La Gruta and Turner, 2014).

Another important T cell subset in pigs is T helper/memory cells (CD3⁺CD4⁺CD8α⁺) (Zuckermann, 1999) which possesses cytolytic function and also secretes IFNγ. The protective response against pseudorabies virus infection has been attributed to the increased frequency of T helper/memory cells (Zuckermann, 1999; De Bruin et al., 2000). The frequency of T helper/memory cells in TBLN-MNCs was significantly enhanced in CNPs-KAg vaccinated pigs. Thus, both T helper/memory and CTLs appear to contribute substantially in improving the cross-protective cellular immune response in pigs vaccinated with chitosan-based influenza nanovaccine.

In conclusion, the mucoadhesive chitosan based IAV nanovaccine formulation delivered as intranasal mist augmented cross-reactive T and B lymphocytes response in pigs at both mucosal (upper and lower respiratory tract and regional lymph nodes-TBLN) and systemic (blood) sites by augmenting secretary IgA, systemic IgG and T cell responses against highly variant IAVs. This augmented virus-specific cross-reactive immune response resulted in reduced nasal virus shedding, reduced viral titers in the pulmonary parenchyma and relatively reduced inflammatory changes in the lungs. Thus, this study indicates that chitosan IAV nanovaccine is a superior vaccine for use against constantly evolving influenza infections in swine herds.

### REFERENCES CITED IN THIS EXAMPLE

Ali, A., Khatri, M., Wang, L., Saif, Y. M., and Lee, C. W. (2012). Identification of swine H1N2/pandemic H1N1 reassortant influenza virus in pigs, United States. Vet Microbiol 158, 60-68.
Amidi, M., Romeijn, S. G., Verhoef, J. C., Junginger, H. E., Bungener, L., Huckriede, A., Crommelin, D. J., and Jiskoot, W. (2007). N-trimethyl chitosan (TMC) nanoparticles loaded with influenza subunit antigen for intranasal vaccination: biological properties and immunogenicity in a mouse model. Vaccine 25, 144-153.
Artursson, P., Lindmark, T., Davis, S. S., and Illum, L. (1994). Effect of chitosan on the permeability of monolayers of intestinal epithelial cells (Caco-2). Pharm Res 11, 1358-1361.
Astete, C. E., and Sabliov, C. M. (2006). Synthesis and characterization of PLGA nanoparticles. J Biomater Sci Polym Ed 17, 247-289.
Babai, I., Samira, S., Barenholz, Y., Zakay-Rones, Z., and Kedar, E. (1999). A novel influenza subunit vaccine composed of liposome-encapsulated haemagglutinin/neuraminidase and IL-2 or GM-CSF. II. Induction of TH1 and TH2 responses in mice. Vaccine 17, 1239-1250.
Bacon, A., Makin, J., Sizer, P. J., Jabbal-Gill, I., Hinchcliffe, M., Illum, L., Chatfield, S., and Roberts, M. (2000). Carbohydrate biopolymers enhance antibody responses to mucosally delivered vaccine antigens. Infect Immun 68, 5764-5770.
Bergquist, C., Johansson, E. L., Lagergard, T., Holmgren, J., and Rudin, A. (1997). Intranasal vaccination of humans with recombinant cholera toxin B subunit induces systemic and local antibody responses in the upper respiratory tract and the vagina. Infect Immun 65, 2676-2684.
Borges, O., Cordeiro-Da-Silva, A., Tavares, J., Santarem, N., De Sousa, A., Borchard, G., and Junginger, H. E. (2008). Immune response by nasal delivery of hepatitis B surface antigen and codelivery of a CpG ODN in alginate coated chitosan nanoparticles. Eur J Pharm Biopharm 69, 405-416.
Calvo, P., Remunan-Lopez, C., Vila-Jato, J. L., and Alonso, M. J. (1997). Chitosan and chitosan/ethylene oxide-propylene oxide block copolymer nanoparticles as novel carriers for proteins and vaccines. Pharm Res 14, 1431-1436.
Carroll, E. C., Jin, L., Mori, A., Munoz-Wolf, N., Oleszycka, E., Moran, H. B. T., Mansouri, S., Mcentee, C. P., Lambe, E., Agger, E. M., Andersen, P., Cunningham, C., Hertzog, P., Fitzgerald, K. A., Bowie, A. G., and Lavelle, E. C. (2016). The Vaccine Adjuvant Chitosan Promotes Cellular Immunity via DNA Sensor cGAS-STING-Dependent Induction of Type I Interferons. Immunity 44, 597-608.
Crisci, E., Mussa, T., Fraile, L., and Montoya, M. (2013). Review: influenza virus in pigs. Mol Immunol 55, 200-211.
De Bruin, T. G., Van Rooij, E. M., De Visser, Y. E., and Bianchi, A. T. (2000). Cytolytic function for pseudorabies virus-stimulated porcine CD4+ CD8dull+ lymphocytes. Viral Immunol 13, 511-520.
Debnath, S. K., Saisivam, S., Debanth, M., and Omri, A. (2018). Development and evaluation of Chitosan nanoparticles based dry powder inhalation formulations of Prothionamide. PLoS One 13, e0190976.
Dhakal, S., Goodman, J., Bondra, K., Lakshmanappa, Y. S., Hiremath, J., Shyu, D. L., Ouyang, K., Kang, K. I., Krakowka, S., Wannemuehler, M. J., Won Lee, C., Narasimhan, B., and Renukaradhya, G. J. (2017a). Polyanhydride nanovaccine against swine influenza virus in pigs. Vaccine 35, 1124-1131.
Dhakal, S., Hiremath, J., Bondra, K., Lakshmanappa, Y. S., Shyu, D. L., Ouyang, K., Kang, K. I., Binjawadagi, B., Goodman, J., Tabynov, K., Krakowka, S., Narasimhan, B., Lee, C. W., and Renukaradhya, G. J. (2017b). Biodegradable nanoparticle delivery of inactivated swine influenza virus vaccine provides heterologous cell-mediated immune response in pigs. J Control Release 247, 194-205.
Dodane, V., Amin Khan, M., and Merwin, J. R. (1999). Effect of chitosan on epithelial permeability and structure. Int J Pharm 182, 21-32.
Dwivedi, V., Manickam, C., Dhakal, S., Binjawadagi, B., Ouyang, K., Hiremath, J., Khatri, M., Hague, J. G., Lee, C. W., and Renukaradhya, G. J. (2016). Adjuvant effects of invariant NKT cell ligand potentiates the innate and adaptive immunity to an inactivated H1N1 swine influenza virus vaccine in pigs. Vet Microbiol 186, 157-163.
Dykhuis, H. C., Painter, T., Fangman, T., and D, H. (Year). “Assessing production parameters and economic impact of swine influenza, PRRS and Mycoplasma hyopneimoniae on finishing pigs in a large production system”, in: Proceedings of AASV Annual Meeting), 75-76.
Etchart, N., Wild, F., and Kaiserlian, D. (1996). Mucosal and systemic immune responses to measles virus haemagglutinin in mice immunized with a recombinant vaccinia virus. J Gen Virol 77 (Pt 10), 2471-2478.
Farrar, M. A., and Schreiber, R. D. (1993). The molecular cell biology of interferon-gamma and its receptor. Annu Rev Immunol 11, 571-611.
Fernandez-Urrusuno, R., Calvo, P., Remunan-Lopez, C., Vila-Jato, J. L., and Alonso, M. J. (1999). Enhancement of nasal absorption of insulin using chitosan nanoparticles. Pharm Res 16, 1576-1581.
Figueiredo, L., Cadete, A., Goncalves, L. M., Corvo, M. L., and Almeida, A. J. (2012). Intranasal immunisation of mice against Streptococcus equi using positively charged nanoparticulate carrier systems. Vaccine 30, 6551-6558.
Foged, C., Brodin, B., Frokjaer, S., and Sundblad, A. (2005). Particle size and surface charge affect particle uptake by human dendritic cells in an in vitro model. Int J Pharm 298, 315-322.
Hiremath, J., Kang, K. I., Xia, M., Elaish, M., Binjawadagi, B., Ouyang, K., Dhakal, S., Arcos, J., Torrelles, J. B., Jiang, X., Lee, C. W., and Renukaradhya, G. J. (2016). Entrapment of H1N1 Influenza Virus Derived Conserved Peptides in PLGA Nanoparticles Enhances T Cell Response and Vaccine Efficacy in Pigs. PLoS One 11, e0151922.
Illum, L., Farraj, N. F., and Davis, S. S. (1994). Chitosan as a novel nasal delivery system for peptide drugs. Pharm Res 11, 1186-1189.
Illum, L., Jabbal-Gill, I., Hinchcliffe, M., Fisher, A. N., and Davis, S. S. (2001). Chitosan as a novel nasal delivery system for vaccines. Adv Drug Deliv Rev 51, 81-96.
Ito, T., Couceiro, J. N., Kelm, S., Baum, L. G., Krauss, S., Castrucci, M. R., Donatelli, I., Kida, H., Paulson, J. C., Webster, R. G., and Kawaoka, Y. (1998). Molecular basis for the generation in pigs of influenza A viruses with pandemic potential. J Virol 72, 7367-7373.
Jabbal-Gill, I., Fisher, A. N., Rappuoli, R., Davis, S. S., and Illum, L. (1998). Stimulation of mucosal and systemic antibody responses against Bordetella pertussis filamentous haemagglutinin and recombinant pertussis toxin after nasal administration with chitosan in mice. Vaccine 16, 2039-2046.
Janke, B. H. (2013). Clinicopathological features of Swine influenza. Curr Top Microbiol Immunol 370, 69-83.
Kim, S. H., and Jang, Y. S. (2017). The development of mucosal vaccines for both mucosal and systemic immune induction and the roles played by adjuvants. Clin Exp Vaccine Res 6, 15-21.
Koppolu, B., and Zaharoff, D. A. (2013). The effect of antigen encapsulation in chitosan particles on uptake, activation and presentation by antigen presenting cells. Biomaterials 34, 2359-2369.
La Gruta, N. L., and Turner, S. J. (2014). T cell mediated immunity to influenza: mechanisms of viral control. Trends Immunol 35, 396-402.
Lebre, F., Borchard, G., Faneca, H., Pedroso De Lima, M. C., and Borges, O. (2016). Intranasal Administration of Novel Chitosan Nanoparticle/DNA Complexes Induces Antibody Response to Hepatitis B Surface Antigen in Mice. Mol Pharm 13, 472-482.
Liu, Q., Zheng, X., Zhang, C., Shao, X., Zhang, X., Zhang, Q., and Jiang, X. (2015). Conjugating influenza a (H1N1) antigen to n-trimethylaminoethylmethacrylate chitosan nanoparticles improves the immunogenicity of the antigen after nasal administration. J Med Virol 87, 1807-1815.
Livak, K. J., and Schmittgen, T. D. (2001). Analysis of relative gene expression data using real-time quantitative PCR and the 2(-Delta Delta C(T)) Method. Methods 25, 402-408.
Lu, F., Mencia, A., Bi, L., Taylor, A., Yao, Y., and Hogenesch, H. (2015). Dendrimer-like alpha-d-glucan nanoparticles activate dendritic cells and are effective vaccine adjuvants. J Control Release 204, 51-59.
Mahapatro, A., and Singh, D. K. (2011). Biodegradable nanoparticles are excellent vehicle for site directed in-vivo delivery of drugs and vaccines. Journal of Nanobiotechnology 9, 1-11.
Mcneela, E. A., O'connor, D., Jabbal-Gill, I., Illum, L., Davis, S. S., Pizza, M., Peppoloni, S., Rappuoli, R., and Mills, K. H. (2000). A mucosal vaccine against diphtheria: formulation of cross reacting material (CRM(197)) of diphtheria toxin with chitosan enhances local and systemic antibody and Th2 responses following nasal delivery. Vaccine 19, 1188-1198.
Meurens, F., Berri, M., Auray, G., Melo, S., Levast, B., Virlogeux-Payant, I., Chevaleyre, C., Gerdts, V., and Salmon, H. (2009). Early immune response following Salmonella enterica subspecies enterica serovar Typhimurium infection in porcine jejunal gut loops. Vet Res 40, 5.
Mirzaei, F., Mohammadpour Dounighi, N., Avadi, M. R., and Rezayat, M. (2017). A New Approach to Antivenom Preparation Using Chitosan Nanoparticles Containing EchisCarinatus Venom as A Novel Antigen Delivery System. Iran J Pharm Res 16, 858-867.
Mishra, N., Goyal, A. K., Tiwari, S., Paliwal, R., Paliwal, S. R., Vaidya, B., Mangal, S., Gupta, M., Dube, D., Mehta, A., and Vyas, S. P. (2010). Recent advances in mucosal delivery of vaccines: role of mucoadhesive/biodegradable polymeric carriers. Expert Opin Ther Pat 20, 661-679.
Mori, A., Oleszycka, E., Sharp, F. A., Coleman, M., Ozasa, Y., Singh, M., O'hagan, D. T., Tajber, L., Corrigan, O. I., Mcneela, E. A., and Lavelle, E. C. (2012). The vaccine adjuvant alum inhibits IL-12 by promoting PI3 kinase signaling while chitosan does not inhibit IL-12 and enhances Th1 and Th17 responses. Eur J Immunol 42, 2709-2719.
Nedumpun, T., Ritprajak, P., and Suradhat, S. (2016). Generation of potent porcine monocyte-derived dendritic cells (MoDCs) by modified culture protocol. Vet Immunol Immunopathol 182, 63-68.
Neutra, M. R., and Kozlowski, P. A. (2006). Mucosal vaccines: the promise and the challenge. Nat Rev Immunol 6, 148-158.
Peniche, H., and Peniche, C. (2011). Chitosan nanoparticles: a contribution to nanomedicine. Polymer International 60, 883-889.
Qi, M., Zhang, X. E., Sun, X., Zhang, X., Yao, Y., Liu, S., Chen, Z., Li, W., Zhang, Z., Chen, J., and Cui, Z. (2018). Intranasal Nanovaccine Confers Homo- and Hetero-Subtypic Influenza Protection. Small.
Rampino, A., Borgogna, M., Blasi, P., Bellich, B., and Cesaro, A. (2013). Chitosan nanoparticles: preparation, size evolution and stability. Int J Pharm 455, 219-228.
Renegar, K. B., and Small, P. A., Jr. (1991). Immunoglobulin A mediation of murine nasal anti-influenza virus immunity. J Virol 65, 2146-2148.
Samuel, C. E. (2001). Antiviral actions of interferons. Clin Microbiol Rev 14, 778-809, table of contents.
Satzer, P., Svec, F., Sekot, G., and Jungbauer, A. (2016). Protein adsorption onto nanoparticles induces conformational changes: Particle size dependency, kinetics, and mechanisms. Eng Life Sci 16, 238-246.
Sawaengsak, C., Mori, Y., Yamanishi, K., Mitrevej, A., and Sinchaipanid, N. (2014). Chitosan nanoparticle encapsulated hemagglutinin-split influenza virus mucosal vaccine. AAPS PharmSciTech 15, 317-325.
Schicker, R. S., Rossow, J., Eckel, S., Fisher, N., Bidol, S., Tatham, L., Matthews-Greer, J., Sohner, K., Bowman, A. S., Avrill, J., Forshey, T., Blanton, L., Davis, C. T., Schiltz, J., Skorupski, S., Berman, L., Jang, Y., Bresee, J. S., Lindstrom, S., Trock, S. C., Wentworth, D., Fry, A. M., De Fijter, S., Signs, K., Diorio, M., Olsen, S. J., and Biggerstaff, M. (2016). Outbreak of Influenza A(H3N2) Variant Virus Infections Among Persons Attending Agricultural Fairs Housing Infected Swine—Michigan and Ohio, July-August 2016. MMWR Morb Mortal Wkly Rep 65, 1157-1160.
Tamura, S., and Kurata, T. (2004). Defense mechanisms against influenza virus infection in the respiratory tract mucosa. Jpn J Infect Dis 57, 236-247.
Torres, M. P., Vogel, B. M., Narasimhan, B., and Mallapragada, S. K. (2006). Synthesis and characterization of novel polyanhydrides with tailored erosion mechanisms. J Biomed Med Res A 76, 102-110.
Van Der Lubben, I. M., Verhoef, J. C., Borchard, G., and Junginger, H. E. (2001). Chitosan for mucosal vaccination. Adv Drug Deliv Rev 52, 139-144.
Van Reeth, K., and Ma, W. (2013). Swine influenza virus vaccines: to change or not to change-that's the question. Curr Top Microbiol Immunol 370, 173-200.
Van Riet, E., Ainai, A., Suzuki, T., and Hasegawa, H. (2012). Mucosal IgA responses in influenza virus infections; thoughts for vaccine design. Vaccine 30, 5893-5900.
Vila, A., Sanchez, A., Janes, K., Behrens, I., Kissel, T., Vila Jato, J. L., and Alonso, M. J. (2004). Low molecular weight chitosan nanoparticles as new carriers for nasal vaccine delivery in mice. Eur J Pharm Biopharm 57, 123-131.
Vincent, A., Awada, L., Brown, I., Chen, H., Claes, F., Dauphin, G., Donis, R., Culhane, M., Hamilton, K., Lewis, N., Mumford, E., Nguyen, T., Parchariyanon, S., Pasick, J., Pavade, G., Pereda, A., Peiris, M., Saito, T., Swenson, S., Van Reeth, K., Webby, R., Wong, F., and Ciacci-Zanella, J. (2014). Review of influenza A virus in swine worldwide: a call for increased surveillance and research. Zoonoses Public Health 61, 4-17.
Vincent, A. L., Ma, W., Lager, K. M., Janke, B. H., and Richt, J. A. (2008). Swine influenza viruses a North American perspective. Adv Virus Res 72, 127-154.
Vincent, A. L., Perez, D. R., Rajao, D., Anderson, T. K., Abente, E. J., Walia, R. R., and Lewis, N. S. (2017). Influenza A virus vaccines for swine. Vet Microbiol 206, 35-44.
Wang, D., Christopher, M. E., Nagata, L. P., Zabielski, M. A., Li, H., Wong, J. P., and Samuel, J. (2004). Intranasal immunization with liposome-encapsulated plasmid DNA encoding influenza virus hemagglutinin elicits mucosal, cellular and humoral immune responses. J Clin Virol 31 Suppl 1, S99-106.
Wang, J. J., Zeng, Z. W., Xiao, R. Z., Xie, T., Zhou, G. L., Zhan, X. R., and Wang, S. L. (2011). Recent advances of chitosan nanoparticles as drug carriers. Int J Nanomedicine 6, 765-774.
Wesley, R. D., and Lager, K. M. (2006). Overcoming maternal antibody interference by vaccination with human adenovirus 5 recombinant viruses expressing the hemagglutinin and the nucleoprotein of swine influenza virus. Vet Microbiol 118, 67-75.
Xu, J., Dai, W., Wang, Z., Chen, B., Li, Z., and Fan, X. (2011). Intranasal vaccination with chitosan-DNA nanoparticles expressing pneumococcal surface antigen a protects mice against nasopharyngeal colonization by Streptococcus pneumoniae. Clin Vaccine Immunol 18, 75-81.
Xu, W., Shen, Y., Jiang, Z., Wang, Y., Chu, Y., and Xiong, S. (2004). Intranasal delivery of chitosan-DNA vaccine generates mucosal SIgA and anti-CVB3 protection. Vaccine 22, 3603-3612.
Yassine, H. M., Al-Natour, M. Q., Lee, C. W., and Saif, Y. M. (2007). Interspecies and intraspecies transmission of triple reassortant H3N2 influenza A viruses. Virol J 4, 129.
Yassine, H. M., Khatri, M., Zhang, Y. J., Lee, C. W., Byrum, B. A., O'quin, J., Smith, K. A., and Saif, Y. M. (2009). Characterization of triple reassortant H1N1 influenza A viruses from swine in Ohio. Vet Microbiol 139, 132-139.
Yu, W., and Xie, H. (2012). A Review on Nanofluids: Preparation, Stability Mechanisms, and Applications.
Zhang, F., Peng, B., Chang, H., Zhang, R., Lu, F., Wang, F., Fang, F., and Chen, Z. (2016). Intranasal Immunization of Mice to Avoid Interference of Maternal Antibody against H5N1 Infection. PLoS One 11, e0157041.
Zhao, K., Chen, G., Shi, X. M., Gao, T. T., Li, W., Zhao, Y., Zhang, F. Q., Wu, J., Cui, X., and Wang, Y. F. (2012). Preparation and efficacy of a live newcastle disease virus vaccine encapsulated in chitosan nanoparticles. PLoS One 7, e53314.
Zuckermann, F. A. (1999). Extrathymic CD4/CD8 double positive T cells. Vet Immunol Immunopathol 72, 55-66.

Publications cited herein are hereby specifically incorporated by reference in their entireties and at least for the material for which they are cited.

It should be understood that while the present disclosure has been provided in detail with respect to certain illustrative and specific aspects thereof, it should not be considered limited to such, as numerous modifications are possible without departing from the broad spirit and scope of the present disclosure as defined in the appended claims. It is, therefore, intended that the appended claims cover all such equivalent variations as fall within the true spirit and scope of the invention.

1. A nanoparticle comprising chitosan and an inactivated influenza A virus (IAV) antigen, wherein the chitosan encapsulates the inactivated IAV antigen.

2. The nanoparticle of claim 1, further comprising tripolyphosphate.

3. The nanoparticle of claim 1, wherein the nanoparticle has a diameter of 500 nm or less.

4. The nanoparticle of claim 1, wherein the nanoparticle has an encapsulation efficiency of inactivated IAV antigen of at least 60%.

5. The nanoparticle of claim 1, wherein the inactivated IAV antigen comprises an inactivated swine IAV antigen.

6. The nanoparticle of claim 1, wherein the inactivated IAV antigen is from an H1N1, H1N2 or H3N2 virus.

7. The nanoparticle of claim 1, wherein the inactivated IAV antigen is homologous, heterologous, or heterosubtypic to an influenza A virus.

8. The nanoparticle of claim 7, wherein the nanoparticle reduces transmission of the influenza A virus.

9. The nanoparticle of claim 7, wherein the nanoparticle reduces nasal shedding of the influenza A virus compared to a control.

10. The nanoparticle of claim 9, wherein the nanoparticle reduces the amount of the influenza A virus in an upper respiratory tract of a subject by at least 1×10¹ TCID₅₀/mL compared to a control.

11. The nanoparticle of claim 9, wherein the reduction in the amount of the influenza A virus occurs within four days of exposure to the influenza A virus.

12. The nanoparticle of claim 1, wherein the nanoparticle elicits an increased amount of IgA antibody in a subject compared to a control.

13. The nanoparticle of claim 12, wherein the increased amount of IgA antibody occurs in mucosa.

14. The nanoparticle of claim 12, wherein the nanoparticle elicits at least 50% more IgA antibody in a subject compared to a control.

15. The nanoparticle of claim 1, wherein the influenza A virus comprises an H1N1, H1N2 or H3N2 influenza A virus.

16. A vaccine comprising a composition of claim 1, in a pharmaceutically acceptable carrier.

17. A method of reducing transmission of an influenza A virus in a subject compared to a control comprising administering to the subject a nanoparticle comprising chitosan and an inactivated influenza A virus (IAV) antigen, wherein the chitosan encapsulates the inactivated IAV antigen.

18. The method of claim 17, wherein the method reduces nasal shedding of the influenza A virus compared to a control.

19. The method of claim 17, wherein the method reduces the amount of the influenza A virus in an upper respiratory tract of the subject by at least 1×10¹ TCID₅₀/mL compared to a control.

20. (canceled)

21. (canceled)

22. (canceled)

23. A method of eliciting an immune response against swine influenza A virus in a subject comprising administering to the subject a nanoparticle comprising chitosan and an inactivated influenza A virus (IAV) antigen, wherein the chitosan encapsulates the inactivated IAV antigen.

24.-28. (canceled)
